(19) United States
(12) Patent Application Publication
(10) Pub. No.: US 2023/0138395 A1
(43) Pub. Date: May 4, 2023

(54) PHARMACEUTICAL COMPOSITIONS OF A THERAPEUTIC POLYENE MACROLIDE AND METHODS OF THEIR USE

(71) Applicant: Biosergen AS, 7075 Tiller Trondheim (NO)

(72) Inventors: Peder M. ANDERSEN, Copenhagen (DK); Yrr MØRCH, Trondheim (NO); Torsten GOESCH, Singapore (SG); Håvard SLETTA, Trondheim (NO); Kristin DEGNES, Trondheim (NO); Andreas ÅSLUND, Trondheim (NO); Peter MOLESWORTH, Trondheim (NO); Heidi JOHNSEN, Trondheim (NO); Ruth SCHMID, Trondheim (NO); Eugenia SANDRU, Trondheim (NO); Sven Even BORGOS, Trondheim (NO); Sofie SNIPSTAD, Trondheim (NO); Einar SULHEIM, Trondheim (NO)

(21) Appl. No.: 17/905,081

(22) PCT Filed: Feb. 26, 2021
(86) PCT No.: PCT/EP2021/054914

§ 371 Date: Aug. 26, 2022

Related U.S. Application Data
(60) Provisional application No. 62/981,762, filed on Feb. 26, 2020.

(51) Int. Cl. A61K 31/7048; A61K 9/51

(52) U.S. Cl. CPC A61K 31/7048; A61K 9/5192; A61K 9/5153; A61K 9/5123; A61K 9/5138

(57) ABSTRACT

Disclosed are pharmaceutical compositions including a plurality of nanoparticles including a compound of the following structure (1) or a pharmaceutically acceptable salt thereof. Also disclosed are methods of their use and preparation.

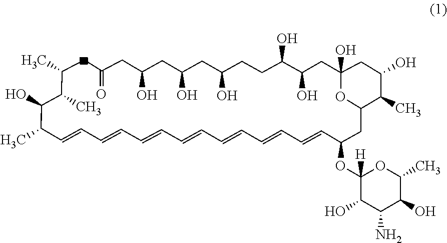

### FIELD OF THE INVENTION

The invention relates to pharmaceutical compositions and methods of their use.

### BACKGROUND

Macrolide antibiotics include a macrocyclic lactone ring bonded to one or more deoxy sugars. Medicinal applications of the macrolide antibiotics are often limited by their restricted shelf life and difficulties in achieving efficient delivery.

The compound of the following structure is a therapeutic polyene macrolide:

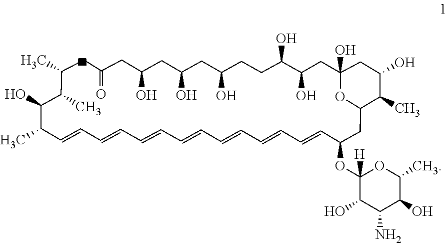 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)CC2O[C@](O)(C[C@@H](O)[C@H](O)CC[C@@H](O)C[C@@H](O)C[C@@H](O)CC(=O)C[C@H]1C)C[C@H](O)[C@H]2C]

There is a need for new formulations including compound 1, or a pharmaceutically acceptable salt thereof.

### SUMMARY OF THE INVENTION

In one aspect, the invention provides pharmaceutical compositions including a plurality of nanoparticles comprising including an active pharmaceutical ingredient that is a compound of the following structure:

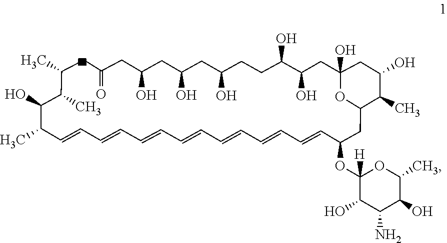 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)CC2O[C@](O)(C[C@@H](O)[C@H](O)CC[C@@H](O)C[C@@H](O)C[C@@H](O)CC(=O)C[C@H]1C)C[C@H](O)[C@H]2C]

or a pharmaceutically acceptable salt thereof.

In some embodiments, the active pharmaceutical ingredient is

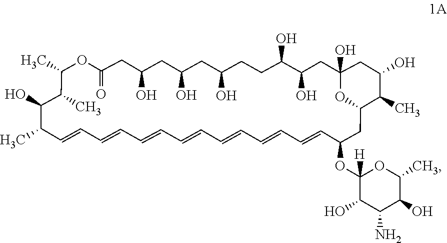 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C]

or a pharmaceutically acceptable salt thereof.

In some embodiments, the pharmaceutical composition further includes a pharmaceutically acceptable polymeric excipient. In some embodiments, the plurality of nanoparticles comprises the pharmaceutically acceptable polymeric excipient. In some embodiments, the active pharmaceutical ingredient is nanoencapsulated. In some embodiments, the pharmaceutically acceptable polymeric excipient is a poly(alkyl cyanoacrylate) or a polyphosphazene. In some embodiments, the pharmaceutically acceptable polymeric excipient is a poly(alkyl cyanoacrylate). In some embodiments, the pharmaceutically acceptable polymeric excipient is a poly(ethylhexyl cyanoacrylate), poly(ethyl cyanoacrylate), poly(n-hexyl cyanoacrylate), poly(4-methylpentyl cyanoacrylate), poly(ethylbutyl cyanoacrylate), poly(butyl cyanoacrylate), or poly(octyl cyanoacrylate). In some embodiments, the pharmaceutically acceptable polymeric excipient is a poly(ethylhexyl cyanoacrylate). In some embodiments, the pharmaceutically acceptable polymeric excipient is a poly(lactic-co-glycolic acid). In some embodiments, the pharmaceutically acceptable polymeric excipient is a protein (e.g., casein, albumin (e.g., human serum albumin, bovine serum albumin, or egg albumin), fibroin, gelatin, or a combination thereof). In some embodiments, the weight ratio of the protein to the active pharmaceutical ingredient is 1:1 to 20:1 (e.g., 5:1 to 20:1, 1:1 to 5:1, 5:1 to 10:1, or 10:1 to 20:1).

In some embodiments, the invention provides a pharmaceutical composition comprising a plurality of nanoparticles comprising a poly(ethylhexyl cyanoacrylate) and an active pharmaceutical ingredient that is a compound of the following structure:

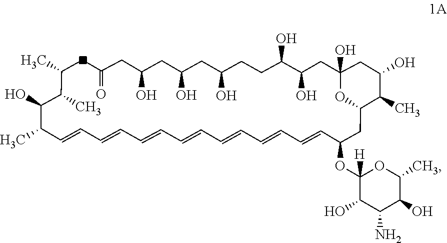 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)C[C@H]1C)C[C@H](O)[C@H]2C]

or a pharmaceutically acceptable salt thereof.

In some embodiments, the invention provides a pharmaceutical composition comprising a plurality of nanoparticles comprising a poly(lactic-co-glycolic acid) and an active pharmaceutical ingredient that is a compound of the following structure:

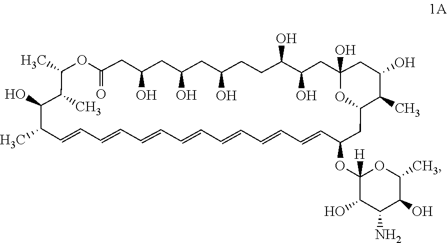 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C]

or a pharmaceutically acceptable salt thereof.

In some embodiments, the invention provides a pharmaceutical composition comprising a plurality of nanoparticles comprising casein, albumin, fibroin, gelatin, or a combination thereof and an active pharmaceutical ingredient that is a compound of the following structure:

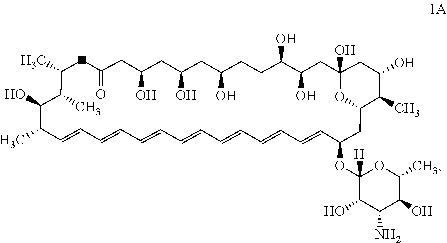 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)C[C@H]1C)C[C@H](O)[C@H]2C]

or a pharmaceutically acceptable salt thereof.

In some embodiments, the pharmaceutically acceptable polymeric excipient is albumin (e.g., human serum albumin, bovine serum albumin, or egg albumin). In some embodiments, the pharmaceutically acceptable polymeric excipient is fibroin. In some embodiments, the pharmaceutically acceptable polymeric excipient is gelatin. In some embodiments, the pharmaceutically acceptable polymeric excipient is casein.

In some embodiments, the pharmaceutical composition is a lyophilized composition. In some embodiments, the pharmaceutical composition further includes a plurality of microbubbles.

In another aspect, the invention provides a pharmaceutical composition comprising a plurality of microbubbles and a plurality of nanoparticles comprising a compound of the following structure:

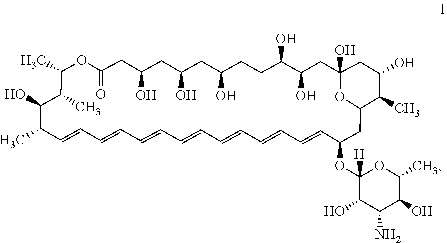 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)CC2O[C@](O)(C[C@@H](O)[C@H](O)CC[C@@H](O)C[C@@H](O)C[C@@H](O)CC(=O)O[C@H]1C)C[C@H](O)[C@H]2C]

or a pharmaceutically acceptable salt thereof.

In some embodiments, the compound is

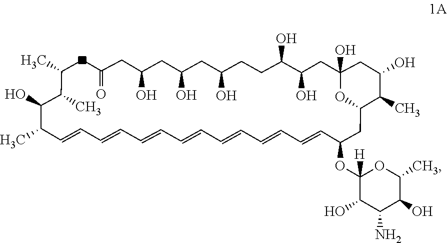 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)C[C@H]1C)C[C@H](O)[C@H]2C]

or a pharmaceutically acceptable salt thereof.

In some embodiments, the nanoparticles comprise a poly(alkyl cyanoacrylate), a polyphosphazene, or a poly(lactic-co-glycolic acid).

In some embodiments, the microbubbles comprise a perfluorocarbon, hydrocarbon, sulfur fluoride gas, air, a component of air, or a mixture thereof. In some embodiments, the microbubbles comprise nitrogen (N₂), oxygen (O₂), argon (Ar), carbon dioxide (CO₂), helium (He), neon (Ne), methane (CH₄), or a mixture thereof. In some embodiments, the microbubbles comprise a perfluorocarbon. In some embodiments, the microbubbles comprise air or a component thereof. In some embodiments, at least a portion of the plurality of nanoparticles is associated with the microbubble surface (e.g., the pharmaceutical composition is a Pickering emulsion).

In some embodiments, the pharmaceutical composition further comprises a surface-active protein (e.g., an albumin (e.g., human serum albumin or bovine serum albumin)). In some embodiments, the pharmaceutical composition comprises 0.1% to 2% (e.g., 0.4% to 0.6%, e.g., 0.5%) (w/w) of a surface-active protein (e.g., an albumin (e.g., human serum albumin or bovine serum albumin)).

In some embodiments, the pharmaceutical composition further comprises a pharmaceutically acceptable surfactant. In some embodiments, the pharmaceutically acceptable surfactant is a non-ionic surfactant. In some embodiments, the pharmaceutically acceptable surfactant is a polyoxyethylene ether, polyoxyethylene fatty acid ester, sorbitan ester, polysorbate, polyethoxylated castor oil, polyoxyethylene/polyoxypropylene block copolymer, or a combination thereof. In some embodiments, the pharmaceutically acceptable surfactant is a polyoxyethylene ether, polyoxyethylene fatty acid ester, or a combination thereof. In some embodiments, the polyoxyethylene fatty acid ester is a polyoxyethylated 12-hydroxystearic acid. In some embodiments, the polyoxyethylene ether is a polyoxyethylene lauryl ether.

In some embodiments, the pharmaceutical composition further comprises a pharmaceutically acceptable stabilizer. In some embodiments, the pharmaceutically acceptable stabilizer is vanillin, butylated hydroxytoluene, butylated hydroxyanisole, or vitamin E. In some embodiments, the pharmaceutically acceptable stabilizer is vanillin. In some embodiments, the pharmaceutical composition comprises 0.1-10% (preferably, 0.5-8%, or, more preferably, 1-5%) (w/w) of a pharmaceutically acceptable stabilizer relative to the particle mass.

In some embodiments, the pharmaceutical composition further comprises a pharmaceutically acceptable oil. In some embodiments, the pharmaceutically acceptable oil is selected from the group consisting of medium chain triglycerides, long chain triglycerides, and combinations thereof. In some embodiments, the pharmaceutically acceptable oil is one or more medium chain triglycerides. In some embodiments, the one or more medium chain triglycerides are selected from the group consisting of Miglyol, Captex, and Kollisolv. In some embodiments, the pharmaceutical composition comprises 0.5-5% (w/w) of a pharmaceutically acceptable oil relative to the particle mass.

In some embodiments, the plurality of nanoparticles has a mean number average diameter of 20-200 nm (preferably, 40-100 nm), as measured by dynamic light scattering. In some embodiments, the plurality of nanoparticles has a mean number average diameter of 30-150 nm (preferably, 80-100 nm), as measured by nanoparticle tracking analysis. In some embodiments, e.g., in pharmaceutical compositions formulated for oral administration, the polydispersity index for the plurality of nanoparticles is 0.5 or less (e.g., 0.3 or less). In some embodiments, e.g., in pharmaceutical compositions formulated for parenteral administration (e.g., intravenous), the polydispersity index for the plurality of nanoparticles is 0.3 or less (e.g., 0.2 or less).

In some embodiments, the pharmaceutical composition is an aqueous composition. In some embodiments, the pH of the pharmaceutical composition is 4.0 to 8.0 (e.g., the pH is 5.0 to 7.0).

In some embodiments, the pharmaceutical composition comprises a co-solvent (e.g., a polar organic solvent). In some embodiments, the polar organic solvent is dimethylsulfoxide, N-methyl-2-pyrrolidone, N,N-dimethylformamide, or a combination thereof.

In certain preferred embodiments, the pharmaceutical composition is an aqueous composition comprising N-methylpyrrolidone and a pharmaceutically acceptable polymeric excipient that is poly(lactic-co-glycolic acid).

In certain preferred embodiments, the pharmaceutical composition is an aqueous composition comprising poly(ethylhexylcyanoacrylate), vanillin, and 6-O-palmitoyl-L-ascorbic acid.

In some embodiments, the pharmaceutical composition is an aqueous composition comprising poly(ethylhexylcyanoacrylate); vanillin; 6-O-palmitoyl-L-ascorbic acid; a polyoxyethylated 12-hydroxystearic acid (e.g., Kolliphor HS 15); a polyoxyethylene lauryl ether (e.g., Brij L23); and medium chain triglycerides (e.g., Miglyol).

In some embodiments, the pharmaceutical composition is an aqueous composition comprising poly(lactic-co-glycolic acid), N-methyl-2-pyrrolidone, and polysorbate.

In some embodiments, the pharmaceutical composition is an aqueous composition comprising poly(lactic-co-glycolic acid), N,N-dimethylformamide, and polyoxyethylene/polyoxypropylene block copolymer.

In some embodiments, the pharmaceutical composition comprises 1-15% (e.g., 2-15%; preferably, 3-10%; more preferably, 3.5-10%; or, yet more preferably, 3-6%) dry (w/w) of the active pharmaceutical ingredient, as measured by liquid chromatography. In some embodiments, the pharmaceutical composition comprises 4.5% to 5.5% dry (w/w) of the active pharmaceutical ingredient, as measured by liquid chromatography. In some embodiments, the pharmaceutical composition comprises 1.5% to 5.5% (e.g., 1.5% to 3.0%) dry (w/w) of the active pharmaceutical ingredient, as measured by liquid chromatography. In some embodiments, the pharmaceutical composition comprises 5% to 10% dry (w/w) of the active pharmaceutical ingredient, as measured by liquid chromatography.

In another aspect, the invention provides a method of treating a subject in need thereof, the method comprising administering to the subject a therapeutically effective amount of the pharmaceutical composition described herein. In another aspect, the invention provides use of a plurality of nanoparticles described herein or a pharmaceutical composition described herein in the manufacture of a medicament for the treatment of a subject in need thereof. In another aspect, the invention provides a pharmaceutical composition described herein for use in the treatment of a subject in need thereof.

In some embodiments, the subject is suffering from a fungal infection caused by Candida, Cryptococcus, Aspergillus, Colletotrichum, Geotrichum, Hormonema, Lecythophora, Paecilomyces, Penicillium, Rhodotorula, Fusarium, Saccharomyces, Trichoderma, Trichophyton, Scopularilopsis, Histoplasma, Blastomyces, or Cocciodioides species. In some embodiments, the subject is suffering from a fungal infection caused by Candida, Aspergillus, or Cryptococcus spp. In some embodiments, the subject is suffering from a fungal infection caused by an azole-resistant Aspergillus sp.

In some embodiments, the pharmaceutical composition is administered intravenously, by inhalation, intranasally, orally, sublingually, buccally, transdermally, intradermally, intramuscularly, intravaginally, parenterally, intra-arterially, intracranially, intrathecally, subcutaneously, intraorbitally, intraventricularly, intraspinally, intraperitoneally, or topically.

In yet another aspect, the invention provides a method of delivering a therapeutically effective amount of compound 1, compound 1A, or a pharmaceutically acceptable salt thereof, to a target site in a subject, the method comprising administering the pharmaceutical composition described herein to the subject. In yet another aspect, the invention provides use of a pharmaceutical composition described herein in the manufacture of a medicament for delivering a therapeutically effective amount of compound 1, compound 1A, or a pharmaceutically acceptable salt thereof, to a target site in a subject. In yet another aspect, the invention provides a pharmaceutical composition described herein for use in delivering a therapeutically effective amount of compound 1, compound 1A, or a pharmaceutically acceptable salt thereof, to a target site in a subject.

In some embodiments, the pharmaceutical composition is administered intravenously. In some embodiments, the target site is the subject's lung.

In still another aspect, the invention provides a method of producing a plurality of nanoparticles comprising a pharmaceutically acceptable polymeric excipient and a compound of the following structure:

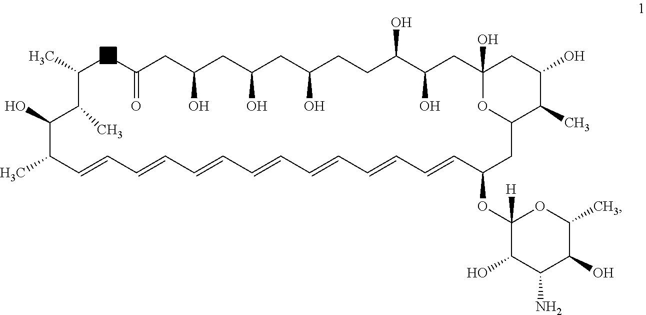 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)CC2O[C@](O)(C[C@@H](O)[C@H](O)CC[C@@H](O)C[C@@H](O)C[C@@H](O)CC(=O)C[C@H]1C)C[C@H](O)[C@H]2C]

or a pharmaceutically acceptable salt thereof;
the method comprising polymerizing a monomeric precursor of the pharmaceutically acceptable polymeric excipient in a liquid comprising the monomeric precursor and the compound or a pharmaceutically acceptable salt thereof, wherein the polymerizing step produces the plurality of nanoparticles.

In some embodiments, the compound is of the following structure:

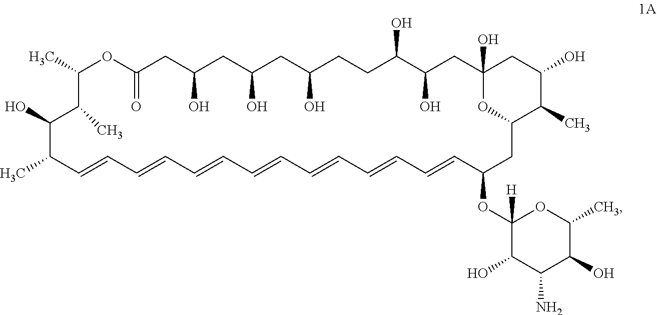 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C]

or a pharmaceutically acceptable salt thereof.

In some embodiments, the liquid further comprises a pharmaceutically acceptable surfactant. In some embodiments, the pharmaceutically acceptable surfactant is a non-ionic surfactant.

In some embodiments, the liquid further comprises a pharmaceutically acceptable stabilizer. In some embodiments, the liquid further comprises a pharmaceutically acceptable oil.

In some embodiments, the monomeric precursor is alkyl cyanoacrylate, and the pharmaceutically acceptable polymeric excipient is poly(alkyl cyanoacrylate).

In some embodiments, the plurality of nanoparticles has a mean number average diameter of 20-200 nm (preferably, 40-100 nm), as measured by dynamic light scattering. In some embodiments, the plurality of nanoparticles has a mean number average diameter of 30-150 nm (preferably, 80-100 nm), as measured by nanoparticle tracking analysis (NTA).

In some embodiments, the liquid is an aqueous composition. In some embodiments, the pH of the liquid is 0.5 to 8.0 (e.g., the pH is 0.5 to 3.0). In some embodiments, the pH of the liquid is 2.0 to 8.0 (preferably, the pH is 3.0 to 7.0).

In some embodiments, the method further includes adding a plurality of microbubbles. In some embodiments, the method further includes lyophilizing the plurality of nanoparticles. In some embodiments, the method further comprises dialyzing the plurality of nanoparticles against deionized water. In some embodiments, the method further comprises adjusting the pH of the liquid to be in the range 4.0 to 8.0 (preferably, in the range 5.0 to 7.0).

In some embodiments, the step of adjusting the pH is performed during a polymerizing step.

### Definitions

The term “about,” as used herein, represents a value that is in the range of ±10% of the value that follows the term “about.”

The term “dry (w/w)” percentage, as used herein, refers to the weight percentage of an ingredient in a composition excluding liquid pharmaceutically acceptable carriers. A dry (w/w) percentage may be measured using, e.g., liquid chromatography.

The term “nanoparticles,” as used herein, represents a population of particles having a Z-average diameter of less than 1000 nm, as measured by dynamic light scattering.

The term “pharmaceutical composition,” as used herein, represents a composition formulated with a pharmaceutically acceptable excipient, and used as part of a therapeutic regimen for the treatment of a disease in a mammal.

The term “pharmaceutical dosage form,” as used herein, represents those pharmaceutical compositions intended for administration to a subject as is without further modification (e.g., without dilution with, suspension in, or dissolution in a liquid solvent).

The term “pharmaceutically acceptable excipient,” as used herein, refers to any ingredient other than the active agent(s) described herein (e.g., a vehicle capable of suspending or dissolving the active agent(s)) and having the properties of being substantially non-toxic and substantially non-inflammatory in a patient. Excipients may include, e.g., antioxidants, disintegrants, dyes (colors), emollients, emulsifiers, fillers (diluents), flavors, fragrances, preservatives, printing inks, sorbents, suspending or dispersing agents, sweeteners, liquid solvents, and buffering agents.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Examples of suitable acids for the formation of such salts include acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic and toluenesulfonic. Glutamate salts are especially preferred.

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject. Non-limiting examples of diseases, disorders, and conditions include fungal infections caused by Candida, Cryptococcus, Aspergillus, Colletotrichum, Geotrichum, Hormonema, Lecythophora, Paecilomyces, Penicillium, Rhodotorula, Fusarium, Saccharomyces, Trichoderma, Trichophyton and Scopularilopsis species. Preferably, the fungal infection is caused by Candida, Aspergillus, or Cryptococcus spp. More preferably, the fungal infection is caused by an azole-resistant Aspergillus sp.

“Treatment” and “treating,” as used herein, refer to the medical management of a subject with the intent to improve, ameliorate, stabilize, prevent or cure a disease, disorder, or condition. This term includes active treatment (treatment directed to improve the disease, disorder, or condition); causal treatment (treatment directed to the cause of the associated disease, disorder, or condition); palliative treatment (treatment designed for the relief of symptoms of the disease, disorder, or condition); preventative treatment (treatment directed to minimizing or partially or completely inhibiting the development of the associated disease, disorder, or condition); and supportive treatment (treatment employed to supplement another therapy).

### BRIEF DESCRIPTION OF THE DRAWINGS

FIG. 1 is a chart showing a survival analysis for mice challenged with Aspergillus fumigatus AF91 and untreated or treated with compound 1A 1.0 mg/kg, compound 1A 0.5 mg/kg, AmBisome 7.5 mg/kg, AmBisome 3.5 mg/kg, voriconazole 7.5 mg/kg, caspofungin 1.0 mg/kg, or vehicle (5% aqueous glucose containing 5% DMSO).

FIG. 2 is a chart showing a survival analysis for mice challenged with Aspergillus fumigatus AF91 and untreated or treated with compound 1A 1.0 mg/kg, compound 1A 0.5 mg/kg, AmBisome 1.0 mg/kg, or AmBisome 0.5 mg/kg.

FIG. 3 is a chart showing a survival analysis for mice challenged with Candida albicans SC5314 and untreated or treated with compound 1A 0.7 mg/kg, compound 1A 0.35 mg/kg, AmBisome 5.4 mg/kg, AmBisome 2.7 mg/kg, voriconazole 4 mg/kg, caspofungin 0.35 mg/kg, fluconazole 6 mg/kg, or vehicle (5% aqueous glucose containing 5% DMSO).

FIG. 4 is a chart showing a survival analysis for mice challenged with Candida albicans SC5314 and untreated or treated with compound 1A 0.7 mg/kg, compound 1A 0.35 mg/kg, AmBisome 0.7 mg/kg, or AmBisome 0.35 mg/kg.

FIG. 5 is a chart showing LC-UV trace for formulation 45, which includes 1.8% (w/w) of Compound 1A. The LC-UV trace demonstrates that an unwanted reaction between Compound 1A and the poly(alkyl cyanoacrylate) (PACA) polymer at retention time 17-19 mins.

FIG. 6 is a chart showing LC-UV trace for formulation 49, which includes 2.4% (w/w) of Compound 1A. The LC-UV trace shows that no degradation or reaction of Compound 1A has occurred in the formulation process. The impurity at 19.5 min is also present in the material used for formulation.

FIG. 7 is a chart showing LC-UV trace for formulation 73, which includes 0.68% (w/w) of Compound 1A. The LC-UV trace shows that no degradation or reaction of Compound 1A has occurred in the formulation process.

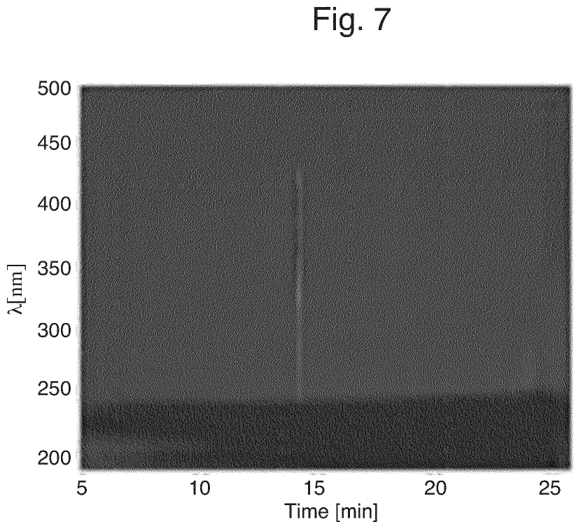

### DETAILED DESCRIPTION

In general, the invention provides pharmaceutical compositions including a plurality of nanoparticles and methods of using the same. The pharmaceutical compositions of the invention include a plurality of nanoparticles including an active pharmaceutical ingredient that is a compound of the following structure:

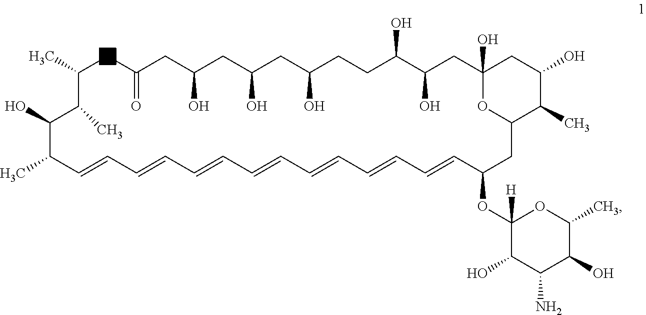 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)CC2O[C@](O)(C[C@@H](O)[C@H](O)CC[C@@H](O)C[C@@H](O)C[C@@H](O)CC(=O)C[C@H]1C)C[C@H](O)[C@H]2C]

or a pharmaceutically acceptable salt thereof.

In some embodiments, the active pharmaceutical ingredient is a compound of the following structure:

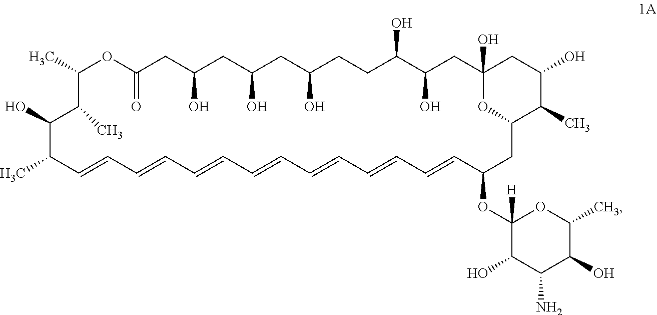 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C]

or a pharmaceutically acceptable salt thereof.

Nanoparticles described herein may include a polymeric excipient (e.g., a polymeric nanoparticle) or may include lipids (e.g., lipid nanoparticles, such as liposomes, micelles, etc.).

Nanoparticles described herein may include a lipid, e.g., a phospholipid (e.g., phosphatidylcholine, phosphatidic acid, phosphatidylserine, phosphatidylethanolamine, or phosphatidylglycerol). In some embodiments, nanoparticles described herein include a phospholipid that is phosphatidylcholine (e.g., dipalmitoylphosphatidylcholine, distearoylphosphatidylcholine, egg phosphatidylcholine and soy phosphatidylcholine) or phosphatidylglycerol (e.g., dipalmitoylphosphatidylglycerol, distearoylphosphatidylglycerol, dilaurylphosphatidylglycerol, or dimyristoylphosphatidylglycerol). For example, the lipids (e.g., phospholipids) may encapsulate the active pharmaceutical ingredient in a vesicle or a micelle.

Preferably, the pharmaceutical composition described herein includes a pharmaceutically acceptable polymeric excipient (e.g., poly(alkylcyanoacrylate), poly(lactic-co-glycolic acid), or a protein (e.g., albumin, fibroin, gelatin, casein, or a combination thereof)). Advantageously, the pharmaceutical compositions described herein may exhibit a commercially acceptable shelf life. Without wishing to be bound by theory, encapsulation of compound 1 (e.g., compound 1A) or a pharmaceutically acceptable salt thereof in the pharmaceutically acceptable polymeric excipient may provide sufficient stability for compound 1 (e.g., compound 1A) or a pharmaceutically acceptable salt thereof to have a commercially acceptable shelf life. For example, the pharmaceutical composition described herein may retain 90% to 110% the label dose of compound 1 (e.g., compound 1A) or a pharmaceutically acceptable salt thereof after being stored at 4° C. for two weeks.

The pharmaceutical compositions described herein may contain at least 2%, preferably at least 3%, and, in particular, at least 5% dry (w/w) of compound 1 or a pharmaceutically acceptable salt thereof (e.g., compound 1A or a pharmaceutically acceptable salt thereof), as measured by liquid chromatography. The pharmaceutical compositions described herein may contain at least 2% (e.g., at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, or at least 9.5%) dry (w/w) of compound 1 or a pharmaceutically acceptable salt thereof (e.g., compound 1A or a pharmaceutically acceptable salt thereof), as measured by liquid chromatography. The pharmaceutical compositions described herein may contain up to 15%, preferably, up to 12%, and, in particular, up to 10% dry (w/w) of compound 1 or a pharmaceutically acceptable salt thereof (e.g., compound 1A or a pharmaceutically acceptable salt thereof), as measured by liquid chromatography. Non-limiting examples of ranges include 2-15%, preferably 3-10%, and, in particular, 3-6% (e.g., 3.5-15%, 4-15%, 4.5-15%, 5-15%, 5.5-15%, 6-15%, 6.5-15%, 7-15%, 7.5-15%, 8-15%, 8.5-15%, 9-15%, 9.5-15%, 3-10%, 3.5-10%, 4-10%, 4.5-10%, 5-10%, 5.5-10%, 6-10%, 6.5-10%, 7-10%, 7.5-10%, 8-10%, 8.5-10%, 9-10%, 9.5-10%, 3-7.5%, 3.5-7.5%, 4-7.5%, 4.5-7.5%, 5-7.5%, 5.5-7.5%, 6-7.5%, 6.5-7.5%, 7-7.5%, 3-5%, 3.5-5%, 4-5%, or 4.5-5%) dry (w/w) of compound 1 or a pharmaceutically acceptable salt thereof (e.g., compound 1A or a pharmaceutically acceptable salt thereof), as measured by liquid chromatography.

The pharmaceutical compositions described herein contain a plurality of nanoparticles. The plurality of nanoparticles may have a mean number average diameter of, e.g., 20-200 nm (preferably, the mean number average diameter is 40-100 nm), as measured by dynamic light scattering. Preferably, the plurality of nanoparticles has a mean number average diameter of 30-150 nm (more preferably, the mean number average diameter is 80-100 nm), as measured by nanoparticle tracking analysis (NTA).

The pharmaceutical compositions described herein include one or more pharmaceutically acceptable excipients, e.g., a pharmaceutically acceptable polymeric excipient, surfactant (e.g., a non-ionic surfactant), stabilizer, carrier (e.g., an oil), and/or flavoring agent.

In the pharmaceutical compositions described herein, polymeric excipients may be used to, e.g., encapsulate an active pharmaceutical ingredient. Non-limiting examples of pharmaceutically acceptable polymeric excipients include poly(alkyl cyanoacrylates), poly(lactic-co-glycolic acid), amphiphilic polyphosphazenes, and proteins. Preferably, the pharmaceutically acceptable polymeric excipient is a poly(alkyl cyanoacrylate) (e.g., poly(ethylhexyl cyanoacrylate), poly(ethyl cyanoacrylate), poly(n-hexyl cyanoacrylate), poly(4-methylpentyl cyanoacrylate), poly(ethylbutyl cyanoacrylate), poly(butyl cyanoacrylate), or poly(octyl cyanoacrylate)). More preferably, the pharmaceutically acceptable polymeric excipient is a poly(ethylhexyl cyanoacrylate) (e.g., poly(2-ethylhexyl cyanoacrylate)). Preferred proteins include albumin, fibroin, gelatin, casein, and combinations thereof.

Nanoparticles including poly(alkyl cyanoacrylate) may be prepared in situ by polymerization of alkyl cyanoacrylate monomers in a composition including compound 1, 1A, or a pharmaceutically acceptable salt thereof. The process of preparing poly(alkyl cyanoacrylate) nanoparticles may include, e.g., customizing the nanoparticular surface by introducing hydrophilic polymers, e.g., including a pharmaceutically acceptable polymeric excipient and, e.g., a surfactant having a reactive moiety capable of reacting with a pharmaceutically acceptable polymeric excipient precursor (e.g., a monomer producing the pharmaceutically acceptable polymeric excipient). Polymerization of the monomer may be initiated using such a surfactant. Alternatively, polymerization of the monomer may be initiated using a polymerization initiator, such as an azo-initiator (e.g., 2,2′-azobis(isobutyronitrile), dimethyl 2,2′-azobis(2-methylpropionate), 2,2′-azobis(4-methoxy-2,4-dimethylvaleronitrile), 2,2′-azobis(2-methylbutyronitrile), 1,1′-azobis(cyclohexane-1-carbonitrile), or 2,2′-azobis(N-butyl-2-methylpropionamide)). In some embodiments, the pharmaceutical composition described herein include nanoparticles including a pharmaceutically acceptable polymeric excipient and an active pharmaceutical ingredient that is compound 1, 1A, or a pharmaceutically acceptable salt thereof. Preferably, the nanoparticles are coated with polyethylene glycol (PEG). Advantageously, a PEG coating on the nanoparticles may reduce clearance, e.g., by the immune system.

Surfactants may be used to stabilize pharmaceutical compositions against, e.g., crystallization and mechanical stresses, such as agitation and/or shearing. A surfactant may be non-ionic or ionic. Non-limiting examples of non-ionic surfactants include a polyoxyethylene ether, polyoxyethylene ester (e.g., polyoxyethylene fatty acid ester), sorbitan ester, polysorbate, sorbitol, ethoxylated phenol, ethoxylated diphenol, polyethoxylated castor oil, polyoxyethylene/polyoxypropylene block copolymer (e.g., poloxamer), poloxamine, fatty acid monoglyceride, fatty acid diglyceride, polysaccharide (e.g., hyaluronic acid or sialic acid), protein (e.g., albumin or casein), and combinations thereof. Preferably, the surfactant is a polyoxyethylene ether or polysorbate. More preferably, the surfactant is a polyoxyethylated 12-hydroxystearic acid (e.g., Kolliphor HS 15), a polyoxyethylene lauryl ether (e.g., Brij L23), or a combination thereof. Non-limiting examples of ionic surfactants include, e.g., sodium dodecyl sulfate, sodium lauryl sulfate, a sulfosuccinate salt, and a fatty acid salt.

A surfactant may be covalently linked to a polymeric excipient. In a non-limiting example, the surfactant described herein may be included in the mixture of the solvent, active agent, and monomers of a polymeric excipient. The surfactant (e.g., a free —OH group in the surfactant) thus may initiate the polymerization of the monomers (e.g., alkyl cyanoacrylate) to encapsulate the active agent in a surfactant-coated nanoparticle (e.g., PEG-coated nanoparticle).

Stabilizers may be used to stabilize pharmaceutical compositions against, e.g., oxidative stress. Non-limiting examples of stabilizers include vanillin, butylated hydroxytoluene, butylated hydroxyanisole, vitamin E, and 6-O-palmitoyl-L-ascorbic acid. Preferably, the stabilizer is vanillin. A pharmaceutical composition may include, e.g., 0.1-10%, preferably, 0.5-8%, and, in particular, 1-5% (e.g., 0.1-9%, 0.1-8%, 0.1-7%, 0.1-6%, 0.1-5%, 0.1-4%, 0.1-3%, 0.1-2%, 0.1-1%, 0.1-0.5%, 0.2-10%, 0.2-9%, 0.2-8%, 0.2-7%, 0.2-6%, 0.2-5%, 0.2-4%, 0.2-3%, 0.2-2%, 0.2-1%, 0.2-0.5%, 0.5-10%, 0.5-9%, 0.5-8%, 0.5-7%, 0.5-6%, 0.5-5%, 0.5-4%, 0.5-3%, 0.5-2%, 0.5-1%, 1-10%, 1-9%, 1-8%, 1-7%, 1-6%, 1-5%, 1-4%, 1-3%, 1-2%, 2-10%, 2-9%, 2-8%, 2-7%, 2-6%, 2-5%, 2-4%, 2-3%, 3-10%, 3-9%, 3-8%, 3-7%, 3-6%, 3-5%, 3-4%, 4-10%, 4-9%, 4-8%, 4-7%, 4-6%, 4-5%, 5-10%, 5-9%, 5-8%, 5-7%, 5-6%, or 5%) (w/w) of the stabilizer relative to the particle mass.

Carriers may be used to suspend or solubilize an active pharmaceutical ingredient in the pharmaceutical composition. Carriers may also be used to prevent Ostwald ripening during the formulation preparation. A suspending or solubilizing carrier may be an aqueous carrier, e.g., water or saline (e.g., isotonic saline). Non-limiting examples of further pharmaceutically acceptable carriers include a pharmaceutically acceptable oil, e.g., medium chain triglycerides, long chain triglycerides, or a combination thereof. Preferably, the pharmaceutically acceptable oil is one or more medium chain triglycerides (e.g., Miglyol, Captex, and Kollisolv). A pharmaceutical composition may include, e.g., 0.5-5%, preferably, 1-5%, and, in particular, 2-3% (e.g., 0.5-5%, 0.5-4.5%, 0.5-4%, 0.5-3.5%, 0.5-3%, 0.5-2.5%, 0.5-2%, 0.5-1.5%, 0.5-1%, 1-5%, 1-4.5%, 1-4%, 1-3.5%, 1-3%, 1-2.5%, 1-2%, 1-1.5%, 1.5-5%, 1.5-4.5%, 1.5-4%, 1.5-3.5%, 1.5-3%, 1.5-2.5%, 1.5-2%, 2-5%, 2-4.5%, 2-4%, 2-3.5%, 2-3%, 2-2.5%, 2.5-5%, 2.5-4.5%, 2.5- 4%, 2.5-3.5%, 2.5-3%, 3-5%, 3-4.5%, 3-4%, 3-3.5%, 3.5-5%, 3.5-4.5%, 3.5-4%, 4-5%, 4-4.5%, or 4.5-5%) (w/w) of the pharmaceutically acceptable oil relative to the particle mass.

For orally administered pharmaceutical compositions, a flavoring agent can be included to make them more palatable. Any effective flavoring agent may be used. The flavoring agents may be natural, artificial, or a mixture thereof. The flavoring agent gives a flavor that is will help to reduce the undesirable taste of the active ingredient. In one embodiment, the flavoring agent may give the flavor of mint, menthol, honey lemon, orange, lemon lime, grape, cranberry, vanilla berry, bubble gum, or cherry. The flavoring agent can be natural or artificial sweetener, e.g., as sucrose, Magnasweet, sucralose, xylitol, sodium saccharin, cyclamate, aspartame, acesulfame, and salts thereof.

The pharmaceutical compositions described herein may be aqueous compositions (e.g., suspensions). The pH of the pharmaceutical composition may be, e.g., 4.0 to 8.0 (preferably 5.0 to 7.0). Alternatively, the pharmaceutical composition may a lyophilized composition. A lyophilized composition may be reconstituted to produce an aqueous composition prior to use.

The pharmaceutical compositions described herein may be used to treat a subject in need thereof. The method of treating the subject includes administering to the subject a therapeutically effective amount of the pharmaceutical composition described herein. The subject may be suffering from a fungal infection (e.g., an invasive fungal infection), e.g., caused by Candida, Cryptococcus, Aspergillus, Colletotrichum, Geotrichum, Hormonema, Lecythophora, Paecilomyces, Penicillium, Rhodotorula, Fusarium, Saccharomyces, Trichoderma, Trichophyton, Scopularilopsis, Histoplasma, Blastomyces, or Cocciodioides species. The subject may be suffering from a fungal infection (e.g., an invasive fungal infection) in addition to one or more of chronic obstructive pulmonary disease (COPD), asthma, cystic fibrosis, chronic pulmonary Aspergillosis, recovery from solid organ transplantation, recovery from a hematological transplantation, and immune suppression following cancer chemotherapy. Preferably, the fungal infection is caused by Candida, Aspergillus, or Cryptococcus spp. More preferably, the fungal infection is caused by an azole-resistant Aspergillus sp.

The pharmaceutical compositions described herein may be administered to the subject in a single dose or in multiple doses. When multiple doses are administered, the doses may be separated from one another by, for example, 1-24 hours, 1-7 days, 1-4 weeks, or 1-12 months. The pharmaceutical composition may be administered according to a schedule, or the pharmaceutical composition may be administered without a predetermined schedule. It is to be understood that, for any particular subject, specific dosage regimes should be adjusted over time according to the individual need and the professional judgment of the person administering or supervising the administration of the pharmaceutical compositions.

While the attending physician ultimately will decide the appropriate amount and dosage regimen, an effective amount of a compound of the invention may be, for example, a total daily dosage of, e.g., between 0.05 mg and 3000 mg of any of the active pharmaceutical ingredient (API) described herein. Alternatively, the dosage amount can be calculated using the body weight of the patient.

In the methods of the invention, the time period during which multiple doses of the pharmaceutical composition are administered to a subject can vary. In some embodiments, doses of the pharmaceutical composition are administered to a subject over a time period that is 1-7 days; 1-12 weeks; or 1-3 months. In some embodiments, the pharmaceutical compositions are administered to the subject over a time period that is, for example, 4-11 months or 1-30 years. In some embodiments, the pharmaceutical compositions are administered to a subject at the onset of symptoms. In any of these embodiments, the amount of the pharmaceutical composition that is administered may vary during the time period of administration. When a pharmaceutical composition is administered daily, administration may occur, for example, 1-12 times per day.

Exemplary routes of administration of the pharmaceutical compositions described herein include intravenous, inhalation, intranasal, oral, sublingual, buccal, transdermal, intradermal, intramuscular, intravaginal, parenteral, intra-arterial, intracranial, intrathecal, subcutaneous, intraorbital, intraventricular, intraspinal, intraperitoneal, and topical administration. Preferably, the route of administration is intravenous.

The pharmaceutical compositions described herein include those formulated for intravenous or intra-arterial administration. The pharmaceutical compositions described herein may include microbubbles and nanoparticles described herein. Preferably, the pharmaceutical composition including microbubbles and nanoparticles are formulated for intravenous administration. Advantageously, pharmaceutical compositions including microbubbles and nanoparticles described herein may facilitate targeted delivery of compound 1 or 1A to a target tissue (e.g., lungs and/or heart).

The pharmaceutical compositions may include microbubbles, e.g., any regular commercially available contrast microbubble, such as Albunex (GE Healthcare), Optison (GE Healthcare), Sonazoid (GE Healthcare), Sonovue (Bracco) or other regular, contrast microbubbles known to the skilled person. In some pharmaceutical compositions, the microbubble surfaces may be associated with nanoparticles. Such microbubbles may be produced, e.g., in a solution of nanoparticles, as described herein. Advantageously, the nanoparticles may have a stabilizing effect on the surface of the microbubbles. The microbubbles may be, e.g., microbubbles filled with a gas or a precursor thereto. The gas may include or may be, e.g., a perfluorocarbon, hydrocarbon (e.g., methane), sulfur fluoride (e.g., SF₆), halogen, air, an air component (e.g., nitrogen (N₂), oxygen (O₂), argon (Ar), carbon dioxide (CO₂), helium (He), or neon (Ne)), or a mixture thereof. Preferably, the gas is a perfluorocarbon, air, an air component (e.g., nitrogen (N₂), oxygen (O₂), argon (Ar), carbon dioxide (CO₂), helium (He), or neon (Ne)), or a mixture thereof. More preferably, the gas is a perfluorocarbon.

Without wishing to be bound by theory, the solubility of the gas in the microbubble may influence the ability of the microbubbles to circulate in the blood and the ability to accumulate in the respiratory system. For example, microbubbles filled with perfluorocarbon gases may have an extended circulation time. Without wishing to be bound by theory, the extended circulation time may be due to the low solubility of perfluorocarbons in blood. Pharmaceutical compositions described herein may include microbubbles including a gas, e.g., perfluorocarbon. Alternatively, the gas may be, e.g., air or a component thereof. Alternatively, the gas may be, e.g., a sulfur fluoride gas, preferably sulfur hexafluoride (SF₆) gas.

Commercially available microbubbles are typically provided as a suspension of gaseous microbubbles stabilized by a shell of lipids, proteins, and/or other surfactants. The microbubbles may be made, e.g., with a surface-active compound, such as a protein, polymer, lipid, surfactant, or a mixture thereof. The surface-active compound(s) may stabilize the microbubble. Preferred, non-limiting examples of the surface-active proteins are albumin (e.g., human or bovine serum albumin or from other suitable biocompatible albumin sources, including synthetic albumin) and casein. Preferred, non-limiting examples of the surface-active lipids are phospholipids. The microbubbles may also contain, e.g., additional stabilizing agents and excipients, e.g., cholesterol or polyoxyethylene-polyoxypropylene.

The pharmaceutical composition described herein can may include, e.g., a modifying agent. The modifying agent may modify the interaction between the components of the pharmaceutical composition. The modifying agent may form complexes or cross-links between the microbubbles and/or the surface-active compounds and the nanoparticles, e.g., thereby increasing the stability of the pharmaceutical composition. The modifying agent may introduce interactions, e.g., between the surface-active compound and the nanoparticles. The modifying agent may be, e.g., urea (H₂N—CO—NH₂). Preferably, the pharmaceutical composition includes a protein (preferably, casein) as a surface-active compound and urea as a modifying agent. Urea may serve as a denaturant for protein. Without wishing to be bound by theory, it is believed that urea may interfere with the hydrogen bonds involved in protein folding. Urea may also form a complex with acid groups on the nanoparticle surface and modify the hydrophilicity of the nanoparticles.

In pharmaceutical compositions including a surface-active compound (e.g., a protein) and urea, the amount of active agent delivered to the targeted tissue is enhanced. By introducing stronger interactions between the surface-active compounds and the nanoparticles, urea may stabilize microbubbles. Without being bound by theory, it is believed that the modification of the microbubbles, the surface-active compound and/or the nanoparticles may enhance the stability of the association between microbubbles and nanoparticles in the pharmaceutical composition. As the association between microbubbles and nanoparticles is enhanced, the number of nanoparticles delivered to the targeted lung tissue may be greater than for compositions lacking the agent enhancing the association between microbubbles and nanoparticles.

The pharmaceutical compositions described herein include microbubbles and nanoparticles may be Pickering emulsions. Hydrophobic solid particles may adsorb strongly at the interface between immiscible fluids, e.g., oil-water, thus forming a Pickering emulsion—an emulsion stabilized by solid nanoparticles or microparticles. Thus, the nanoparticles associated with the microbubble surface may stabilize the composition as a Pickering emulsion. Advantageously, pharmaceutical compositions described herein may be further stabilized by formulation as a Pickering emulsion. The mean diameter of the microbubbles associated with nanoparticles may be, e.g., 0.5 to 30 μm (e.g., 1-10 μm). The microbubble diameters may be measured, e.g., by a two-dimensional analysis of the images of the microbubbles using an ImageJ image analyzer.

Additionally, the pharmaceutical composition described herein may include free nanoparticles, e.g., in addition to the microbubble surface-associate nanoparticles. The pharmaceutical composition described herein may include nanoparticles associated with microbubbles described herein as well as free nanoparticles described herein.

Pharmaceutical compositions including microbubbles and nanoparticles described herein may be prepared, e.g., according to a method including combining gas or microbubbles and nanoparticles described herein. For example, nanoparticles may be in a solution. The nanoparticles may be prepared in situ as described herein, or may be reconstituted from a dry composition.

Pharmaceutical compositions including microbubbles and nanoparticles described herein may be prepared, e.g., according to a method including:
a. Adding microbubbles described herein and nanoparticles described herein to a solution, and
b. Mixing the solution to produce the pharmaceutical composition.

Alternatively, pharmaceutical compositions including microbubbles and nanoparticles described herein may be prepared, e.g., according to a method including:
a. Synthesizing nanoparticles described herein,
b. Combining the nanoparticles and a surface-active compound,
c. Adding gas to the solution, and
d. Mixing the solution to produce the pharmaceutical composition.

In some embodiments, the solution is mixed (e.g., stirred) for 2 seconds to 60 minutes (e.g., 1-10 minutes). Solution mixing methods are known in the art, e.g., ultrasonication, mechanical stirring, microfluidics, shaking, etc. In some preparation methods for microbubble-containing formulations, the composition may be degassed prior to the addition of a microbubble gas. Degassing methods are known in the art; non-limiting examples of the degassing methods include, e.g., sonication and freeze-pump-thaw degassing.

The pharmaceutical compositions described herein include those formulated for oral administration (“oral dosage forms”). Oral dosage forms can be, for example, in the form of tablets, capsules, liquid suspensions, powders, granulates, or pellets, which contain the active pharmaceutical ingredient and one or more pharmaceutically acceptable excipients. These excipients may be, for example, inert diluents or fillers; granulating and disintegrating agents; binding agents; and lubricating agents, glidants, antiadhesives, colorants, flavoring agents, plasticizers, humectants, buffering agents, and the like.

Controlled release compositions for oral use may be constructed to release the active drug by controlling the dissolution and/or the diffusion of the active pharmaceutical ingredient. Dissolution- or diffusion-controlled release can be achieved by appropriate coating of a tablet, capsule, pellet, granulate, or particles containing the API, or by incorporating the particles containing the API into an appropriate matrix. In some embodiments, compositions include biodegradable, pH, and/or temperature-sensitive polymer coatings. For example, an oral dosage form may include an active pharmaceutical ingredient (e.g., nanoparticles described herein).

The pharmaceutical compositions described herein may be prepared using techniques and methods described herein and those known in the art.

The invention further features a method of producing a plurality of nanoparticles including an active pharmaceutical ingredient that is a compound of the following structure:

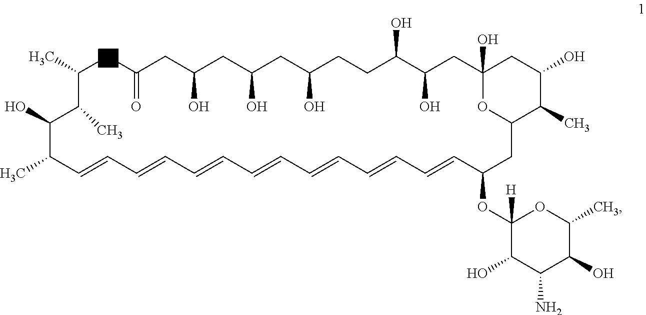 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)CC2O[C@](O)(C[C@@H](O)[C@H](O)CC[C@@H](O)C[C@@H](O)C[C@@H](O)CC(=O)C[C@H]1C)C[C@H](O)[C@H]2C]

or a pharmaceutically acceptable salt thereof.

In particular, the plurality of nanoparticles may include, e.g., a pharmaceutically acceptable polymeric excipient.

The method accordingly includes polymerizing a monomeric precursor of the pharmaceutically acceptable polymeric excipient in a liquid including the monomeric precursor and the compound or a pharmaceutically acceptable salt thereof. The polymerizing step produces the plurality of nanoparticles.

In some embodiments, the active pharmaceutical ingredient is a compound of the following structure:

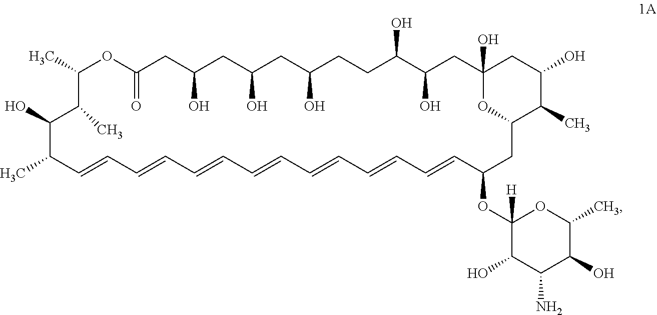 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C]

or a pharmaceutically acceptable salt thereof.

In the production methods described herein, the liquid may further include a pharmaceutically acceptable surfactant (e.g., non-ionic surfactant, such as a polyoxyethylene ether, polyoxyethylene fatty acid ester, sorbitan ester, polysorbate, polyethoxylated castor oil, polyoxyethylene/polyoxypropylene block copolymer, or a combination thereof). Preferably, the pharmaceutically acceptable surfactant is a polyoxyethylene ether (e.g., a polyoxyethylene lauryl ether), polyoxyethylene fatty acid ester (e.g., a polyoxyethylated 12-hydroxystearic acid), or a combination thereof.

In the production methods described herein, the liquid may further include a pharmaceutically acceptable stabilizer (e.g., vanillin, butylated hydroxytoluene, butylated hydroxyanisole, or vitamin E). Preferably, the pharmaceutically acceptable stabilizer is vanillin.

In the production methods described herein, the liquid may further include a pharmaceutically acceptable oil (e.g., an oil selected from the group consisting of medium chain triglycerides, long chain triglycerides, and combinations thereof). Preferably, the pharmaceutically acceptable oil is one or more medium chain triglycerides (e.g., Miglyol, Captex, and Kollisolv).

In the production methods described herein, the monomeric precursor may be, e.g., alkyl cyanoacrylate (e.g., ethylhexyl cyanoacrylate), and the pharmaceutically acceptable polymeric excipient may be, e.g., poly(alkyl cyanoacrylate) (e.g., poly(ethylhexyl cyanoacrylate)).

In the production methods described herein, the plurality of nanoparticles may have a mean number average diameter of 20-200 nm (e.g., 40-100 nm), as measured by dynamic light scattering. In the production methods described herein, the plurality of nanoparticles may have a mean number average diameter of 30-150 nm (e.g., 80-100 nm), as measured by nanoparticle tracking analysis.

The liquid may be, e.g., an aqueous composition (e.g., an aqueous composition having the pH of 0.5 to 8.0 (e.g., pH of 0.5 to 3.0, 2.0 to 8.0, or 3.0 to 7.0)).

The production method may further include the step of lyophilizing the plurality of nanoparticles. Additionally or alternatively, the production method may further include the step of dialyzing the plurality of nanoparticles against deionized water.

In the production methods described herein, pH of the liquid may be adjusted as desired. For example, the pH of the liquid may be adjusted to be in the range 4.0 to 8.0 (e.g., 5.0 to 7.0). In some embodiments, the step of adjusting the pH is performed during the step of polymerizing. In other embodiments, the step of adjusting the pH is performed before or after the step of polymerizing.

The following examples are meant to illustrate the invention. They are not meant to limit the invention in any way.

### EXAMPLES

### Example 1. Preparation of Polyphosphazene Nanoparticulate Compositions

Ethyl 4-aminobenzoate (CAS: 94-09-7) and polyethylene glycol-substituted (8 to 13 repeating units) amphiphilic polyphosphazene (POPZ) polymer with a molecular weight of 7 to 11 kDa (SINTEF) were used. 11 mg of compound 1A (custom made) was dissolved in 11 mL of DMF and 100 mg of POPZ. The solution was added dropwise to 11 mL of distilled water under rigorous stirring at room temperature. The sample was dialyzed against distilled water with one shift. The particle solution was lyophilized to produce dry nanoparticle powder with a theoretical compound 1A loading of 10% (w/w).

### Example 2. Preparation of Poly(2-ethylhexyl cyanoacrylate) Nanoparticulate Compositions at pH=2

An aqueous solution containing PEG stabilizers Kolliphor HS 15 (0.12 g) and BrijL23 (0.12 g) in 12 mL of 0.01M HCl (pH 2) was prepared. A solution containing 0.096 g of compound 1A (custom made) dissolved in 2-ethylhexyl cyanoacrylate (0.8 g) together with stabilizers (0.05 g vanillin and 15 μL Miglyol 812) was prepared and kept on stirring for 2 hours at room temperature.

The two solutions were mixed on ice and homogenized on an ultrasonifier (50% amplitude) for 3 minutes with a 10 second pause every 30 seconds. The nanoemulsion was polymerized at room temperature for three hours on a rotator. The pH was adjusted to pH 6 with 0.1M NaOH, and further polymerized at room temperature overnight (on a rotator). The particle sample was dialyzed against distilled water. The final product was a liquid suspension. The theoretical loading was 10% (w/w).

### Example 3. Preparation of Poly(2-ethylhexyl cyanoacrylate) Nanoparticulate Compositions at pH=4

The same procedure was used for this example as that which is described in Example 2 with the exception that the aqueous solution contained 0.1 mM HCl (pH=4) instead of 0.01M HCl.

### Example 4. Preparation of Poly(2-ethylhexyl cyanoacrylate) Nanoparticulate Compositions at pH=1

The same procedure was used for this example as that which is described in Example 2 with the exception that the aqueous solution contained 0.1 M HCl (pH=1) instead of 0.01M HCl.

### Example 5. Preparation of Poly(2-ethylhexyl cyanoacrylate) Nanoparticulate Compositions at without BrijL23

The same procedure was used for this example as that which is described in Examples 2-4 with the exception that the aqueous solution contained does not contain BrijL23 and the amount of Kolliphor HS 15 is doubled.

### Example 6. Preparation of Poly(2-ethylhexyl cyanoacrylate) Nanoparticulate Compositions with More Vanillin

The same procedure was used for this example as that which is described in Examples 2-5 with the exception that the amount of vanillin was increased to 0.1 g.

### Example 7. Preparation of Poly(2-ethylhexyl cyanoacrylate) Nanoparticulate Compositions with More Vanillin

The same procedure was used for this example as that which is described in Examples 2-5 with the exception that the amount of vanillin was decreased to 0.025 g.

### Example 8. Preparation of Poly(2-ethylhexyl cyanoacrylate) Nanoparticulate Compositions at pH=2, Theoretical Loading=14.8%

The same procedure was used for this example as that which is described in Example 2 with the exception that the theoretical loading of compound 1A was 14.8% (w/w). The measured final loading of compound 1A was 3.3% (w/w).

### Example 9. Preparation of Poly(2-ethylhexyl cyanoacrylate) Nanoparticulate Compositions at pH=3, Theoretical Loading=14.8%

The same procedure was used for this example as that which is described in Example 2 with the exception that (1) the aqueous solution contained 1 mM HCl (pH=3) instead of 0.01M HCl, and (2) the theoretical loading of compound 1A was 14.8% (w/w). The measured final loading of compound 1A was 3.6% (w/w).

### Example 10. Preparation of Poly(2-ethylhexyl cyanoacrylate) Nanoparticulate Compositions at pH=4, Theoretical Loading=14.8%

The same procedure was used for this example as that which is described in Example 3 with the exception that the theoretical loading of compound 1A was 14.8% (w/w). The measured final loading of compound 1A was 3.8% (w/w).

### Example 11. Physicochemical Characterization of Nanoparticulate Compositions

The sizes and size distributions were measured using dynamic light scattering (Malvern Zetasizer and Nanoparticles Tracking Analyzer (NTA)) in phosphate buffer (pH 7). Dry weight was determined by drying three sample aliquots at 50° C. overnight. Drug loading and stability was determined weighing/pipetting three sample aliquots, dissolution in DMSO, and dilution for LC-DAD-QTOF analysis. The concentration of compound 1A was determined using three samples of amphotericin B USP as a standard. The LC-QTOF method was:
Mobile phases: 0.1% formic acid [A] and acetonitrile [B]
HPLC system: Agilent 1290 HPLC system with a 1290 DAD connected to a QTOF
Column: Polaris 3 C18, 150×2 mm, 3 μm (Varian)
Column thermostat: 30° C.
Flow rate: 0.3 ml/min
Injection volume: 2 μL
Wavelength: 385 nm for measuring compound 1A/AMB, Scan 190-600 nm
Software: MassHunter Qualitative analysis B.0.600
Post time: 5 min
QTOF was operated in negative electrospray mode (ESI−)
The HPLC gradient is shown in Table 1.

TABLE 1
HPLC gradient:

| Time (min) | % mobile phase B |
|---|---|
| 0 | 4.8 |
| 20 | 61.8 |
| 22 | 80.0 |
| 22.5 | 4.8 |
| 23.0 | end |

Size and Size Distribution

The nanoparticle size, size distribution, dry weight, and drug loading are summarized in Table 2.

TABLE 2

| | Formulation Example | | |
|---|---|---|---|
| | 1 | 2 | 3 |
| Z-average mean diameter | 143 | 158 | 214 |
| (nm)¹ | | | |
| Mean number average diameter | 85 | 48 | 40 |
| (nm)¹ | | | |
| Size polydispersity index | 0.18 | 0.25 | 0.33 |
| (PDI)¹ | | | |
| Number-average (mode) diameter | 74 | 78 | 82 |
| (nm)² | | | |
| Number-average (mean) diameter | 87 | 103 | 64 |
| (nm)² | | | |
| Dry weight (g) or (mg/mL)³ | 0.071 g | 2.14 mg/mL | 1.04 mg/mL |
| compound 1A loading [% (w/w)] | 1.07 | 2.8 | 3.9 |

¹As measured by Zetasizer;
²As measured by NTA;
³Example 1 particles were lyophilized, and Examples 2 and 3 particles were dialyzed, thereby producing dry material (g) and aqueous suspensions (mg/mL), respectively.

Table 3 provides particle size distribution details measured by NTA.

TABLE 3

| | Formulation Example | | |
|---|---|---|---|
| | 1 | 2 | 3 |
| Mean diameter (nm) | 87.3 | 103 | 82.4 |
| Mode diameter (nm) | 73.5 | 78 | 64.6 |
| Standard Deviation (nm) | 22.2 | 35.8 | 30.6 |
| D10 (nm) | 64 | 71 | 57.4 |
| D50 (nm) | 83.1 | 92.9 | 73.4 |
| D90 (nm) | 114.4 | 152.3 | 118 |

Drug Loading and Stability

In the composition from Example 1, 42 mol % of the polyenes in the particles were compound 1A. The loading of compound 1A was 1.1% (w/w), and the total polyene loading was 2.5% (w/w).

The composition from Example 2 was found to contain the same polyene impurity as in Example 1 but at a lower level, as 10% of the total polyenes corresponded to the same major polyene impurity found in Example 1. The concentration of compound 1A in the liquid sample was 0.59 mg/mL giving a compound 1A loading of 2.8% (w/w) in the particles.

In the composition of Example 3, no significant degradation of compound 1A was observed. The concentration of compound 1A in the liquid sample was 0.40 mg/mL giving the compound 1A loading of 3.9% (w/w) in the particles. While small quantities of minor polyene impurities were observable, their concentrations were too low to determine their masses.

Long-Term Stability of Compound 1A in Poly(2-ethylhexyl cyanoacrylate) Nanoparticles

The Example 3 composition was re-analyzed two weeks after preparation. No degradation due to storage for two weeks at 4° C. was observed by UV at λ=385 nm.

Longer time periods and different temperature and humidity levels may be used to test the shelf life of the compositions.

### Example 12. Efficacy of the Example 3 Composition

Efficacy of the compositions was determined using Minimum Inhibitory Concentration assays, and the concentration of active compound giving 50% growth inhibition of the indicator organism was reported (MIC50). The assay was performed in well plates with two (Müller-Hinton medium) or three parallel (M19-medium) cell cultures for each condition.

Cell Culture Medium: Müller-Hinton and M19 without NaCl.

Indicator Organism: Candida albicans ATCC 10231

Stock Solutions of the Active Compounds and Control:
Amphotericin B (USP): vacuum dried powder was dissolved in DMSO to 2.5 mg/mL giving a final concentration after inoculation of 2.5 μg/mL in the well with the highest concentration.
compound 1A (custom made): powder was dissolved in DMSO to 2.5 mg/mL giving a final concentration after inoculation of 2.5 μg/ml compound 1A in the well with the highest concentration. It is assumed that the batch is 70% pure.
Example 3 composition: the formulation was a suspension with 18 mg/mL nanoparticles. The suspension was diluted in a medium and C. albicans inoculum to a final concentration of 5 μg/mL of compound 1A and 130 μg/mL of poly(2-ethylhexyl cyanoacrylate). From this solution 10 dilutions were prepared giving 0.009 μg/ml compound 1A in the lowest concentration.
Empty poly(ethylbutyl cyanoacrylate) particles: empty poly(ethylbutyl cyanoacrylate) particles were used as a reference. The empty poly(ethylbutyl cyanoacrylate) was produced in the same manner as those in Examples 2 and 3, with the exception that they were prepared at pH 1, with vanillin concentration of 10% (w/w), and. The particles were diluted in the same manner as Example 3. The highest poly(ethylbutyl cyanoacrylate) concentration tested was 130 μg/mL.

Growth measurements were performed at OD600.

As described in Example 11, compound 1A was found to be stable in the Example 3 composition. This material was tested in an in vitro efficacy assay against C. albicans to verify that the active compound is released from the particles at a rate sufficient to inhibit C. albicans to the same extent as pure compound 1A.

The measured MIC50 values were higher in the M19 medium than in the Müller-Hinton medium because the strain grows faster in the M19-medium. The plates were measured manually throughout the night. Both assays in the M19 medium and in the Müller-Hinton medium showed that the MIC50 of the Example 3 composition is 2-2.5× higher than that of pure compound 1A. As control, inhibition of empty poly(ethylbutyl cyanoacrylate) particles were tested in M19 medium. No growth inhibition of Candida albicans ATCC 10231 was observed up to a concentration of 130 μg/mL poly(ethylbutyl cyanoacrylate), which was the highest concentration tested. The highest Example 3 composition concentration tested (5 μg/mL) contained 130 μg/ml poly(2-ethylhexyl cyanoacrylate).

TABLE 4

MIC50 (μg/mL)

| | M19 medium | Müller-Hinton medium |
|---|---|---|
| Amphotericin B | 0.34 | 0.04 |
| Example 3 | 0.38 | 0.15 |
| compound 1A | 0.18 | 0.06 |

### Example 13

Production and characterization of dye-loaded nanoparticles: Other nanoparticles including compound 1 or a pharmaceutically acceptable salt thereof (e.g., compound 1A or a pharmaceutically acceptable salt thereof) are prepared. For example, PEG-coated and dye-loaded nanoparticles of either polymer or lipid are prepared by the miniemulsion process as follows:

Polymeric nanoparticles: An oil phase consisting of an alkyl cyanoacrylate (e.g., n-butyl cyanoacrylate), a mixture of a pharmaceutically acceptable oil (e.g., Miglyol) with added near-infrared dye (e.g., DIR) is prepared. Then, an aqueous phase containing one or more surfactants (e.g., Brij L23 and/or Kolliphor HS 15) is added to the oil phase. Certain surfactants, such as Kolliphor HS 15, may be used as polymerization initiators, thus producing a polymeric excipient (e.g., poly(alkyl cyanoacrylate)) covalently attached to polyethylene glycol. An oil-in-water emulsion is prepared by mixing the oil and aqueous phase. The dispersion is dialyzed (e.g., with a Spectra/Poor dialysis membrane MWCO 100,000 Da) against an aqueous fluid to remove the surfactants not incorporated into nanoparticles.

Lipid nanoparticles: A lipid phase consisting of a mixture of lipids (e.g., stearic acid and isopropyl palmitate) and a mixture of a pharmaceutically acceptable oil (e.g., Miglyol) with added near-infrared dye (e.g., DIR) is pre-heated until melted. A water phase consisting of a distilled water and additives (e.g., surfactants (e.g., lecithin 80H and Andean QDP Ultra)) is prepared. The lipid and water phase are mixed.

Exemplary production and characterization of compound 1A-loaded nanoparticles: PEG-coated and compound 1A-loaded poly(alkyl cyanoacrylate) nanoparticles are prepared by the miniemulsion method as follows: an oil phase containing an alkyl cyanoacrylate (e.g., 2-ethylbutyl cyanoacrylate), a pharmaceutically acceptable oil (e.g., Miglyol) and compound 1A is prepared. An aqueous phase containing surfactants (e.g., Brij L23 and Kolliphor HS 15) is prepared. Kolliphor HS 15 may also serve as a polymerization initiator. An oil-in-water emulsion is prepared by mixing the oil and aqueous phase. The dispersion is dialyzed extensively against an aqueous fluid to remove surfactants not associated with the particles (e.g., with a dialysis membrane, MWCO 100,000 Da).

Dynamic light scattering (e.g., Zetasizer) can be used to determine hydrodynamic diameter, hydrodynamic diameter distribution, and zeta potential. The dry weight content of the final solution (nanoparticle concentration) can be determined after sufficient drying. To calculate the amount of encapsulated drug, drug content is extracted from the particles, and the extracted amount of compound 1A is quantified by using LC-MS/MS method. Dynamic light scattering method typically shows a nanoparticle size (z-average) for drug-loaded nanoparticles.

Production and characterization of nanoparticle-stabilized microbubbles: Gas-filled microbubbles associated with nanoparticles are produced as follows: a solution containing a surface-active compound (e.g., 2% (w/w) casein) is prepared. The compound 1A-loaded PEGylated nanoparticles (e.g., those described in Examples 2, 3, and 8-10 or in this Example) are mixed with the casein solution. The solution is saturated with a gas (e.g., air or perfluoropropane). The vial is sealed under gas-filled atmosphere using septum. In some preparation methods for microbubble-containing formulations, the composition may be degassed prior to the addition of a microbubble gas.

The average size and concentration of the resulting nanoparticle-stabilized microbubbles are determined from light microscopy images using a 20× phase contrast objective and cell counter (hemocytometer). Microbubbles are counted, and the size is calculated by analyzing the images using ImageJ image analyzer.

Fluorescence microscopy (using same type of nanoparticles only encapsulating a fluorescent dye instead of drug) and electron microscopy are used to confirm that nanoparticles are associated with the microbubbles forming a stabilizing (mono)layer.

### Example 14

In this study, the potential of microbubbles associated with nanoparticles for specific drug delivery targeted to the lungs is evaluated, e.g., in healthy animals (e.g., mice). High local concentrations are achieved with gas-filled microbubbles stabilized by nanoparticles.

Methods

The study is designed with one animal in each group. Nanoparticles labelled with a near-infrared fluorescent dye are developed according to the procedure as described in Example 13 and used. Using a whole animal imager, these nanoparticles are localized inside small animals.

In the experiments, if it is necessary to sacrifice a test animal, the animal may be given anesthesia, used for experiments, and sacrificed before waking up. During storage at the animal facility, the animal welfare is monitored, and the animals are given food and water ad libitum.

Animal Experiments:

1. An animal is randomly selected, weighed and given a subcutaneous injection of a solution which provides full anesthesia (e.g., fentanyl/medetomidine/midazolam/water (2:1:2:5)).

2. A catheter is placed, allowing for intravenous injections.

3. The desired bubbles are injected.

4. The animals are left to sleep for the desired time before being euthanized.

5. Lungs, liver, kidney and spleen can then be harvested.

6. Fluorescence from the organs can be imaged using a complete-animal imager (e.g., Pearl).

Nanoparticles:

In order to perform the animal experiments, near-infrared labelled nanoparticles are used.

Following the control containing only nanoparticles, the pharmaceutical composition including nanoparticles associated with microbubbles (e.g., as described in Example 13) are tested, wherein the microbubbles contain a gas (e.g., perfluoropropane).

In order to compare the results directly, the lungs from a plurality of animals (e.g., three) are imaged together.

Microbubbles with Lipid Nanoparticles:

Microbubbles are produced with lipid nanoparticles and tested.

The stability of the lung accumulation is tested to assess whether nanoparticles remain in the lungs or redistribute to other organs. The biodistributions in animals (e.g., two) after desired amounts of time (e.g., 1 h and 2 h after injection) are then examined.

A high local concentration of nanoparticles in the lungs is beneficial for targeted delivery of an active pharmaceutical ingredient to the lung tissue.

### Example 15

In this study, gas-filled microbubbles stabilized by nanoparticles are tested in the targeted drug delivery to the lungs. The study is performed, e.g., in healthy mice.

Methods

Nanoparticles labelled with a fluorescent dye (e.g., near-infrared dye) are prepared as described in Example 13. Using a whole animal imager, these nanoparticles are localized inside small animals.

In the experiments, if it is necessary to sacrifice a test animal, the animal may be given anesthesia, used for experiments, and sacrificed before waking up.

Production of nanoparticle-stabilized microbubbles: gas-filled microbubbles associated with nanoparticles are produced as follows:

A solution containing a surface-active compound (e.g., 2% (w/w) casein) is prepared. The dye-loaded PEGylated NPs are mixed with the casein solution. The solution is saturated with a gas (e.g., sulfur hexafluoride or perfluoropropane). In one batch, a modifying agent (e.g., urea) is added in order to further promote association between microbubbles and nanoparticles. The vial is sealed under gas-filled atmosphere using septum. The animal experiment is performed as described in Example 10. Each animal is intravenously injected with, for example, either:

A) perfluoropropane-filled microbubbles stabilized by poly(2-ethylhexyl cyanoacrylate) nanoparticles.

B) perfluoropropane-filled microbubbles with urea stabilized by poly(2-ethylhexyl cyanoacrylate) nanoparticles.

C) sulfur hexafluoride-filled microbubbles stabilized by poly(2-ethylhexyl cyanoacrylate) nanoparticles.

After the injections, images are taken, and fluorescence intensities are used to assess the biodistribution of drug-filled nanoparticles in the test animal lungs.

### Example 16

The efficacy of compound 1A was tested against a broad panel of yeast and filamentous fungal strains, as measured by minimum inhibitory concentration (MIC) and minimum fungicidal concentration (MFC). Amphotericin B, caspofungin, fluconazole, and voriconazole were tested as comparators.

Materials: Isolates were taken from the culture collection at the Center for Medical Mycology and included 20 strains each of Candida albicans, C. glabrata, C. parapsilosis, C. tropicalis, Cryptococcus neoformans, Aspergillus fumigatus, A. niger, A. terreus, Fusarium spp., and mucormycetes (Rhizopus and Mucor spp.). Also included were 10 strains of each of C. krusei, A. terreus, and Penicillium spp., and 5 strains of Paecilomyces spp. The panel contained 17 Candida strains with known elevated MICs to other current antifungals; recent clinical strains were also included. Testing also included 15 dimorphic fungal isolates.

Minimum Inhibitory Concentration: MIC testing was performed according to the Clinical and Laboratory Standards Institute (CLSI) M27-A3 and M38-A2 standards for the susceptibility testing of yeasts and filamentous fungi, respectively (Cryptococcus isolates were incubated for 72 h). For yeast strains, the incubation temperature and time were 35° C. and 24-48 h, respectively, and the inoculum size was 0.5-2.5×103 CFU/ml. For filamentous strains, the inoculum size was 0.4-5×104 CFU/ml, and the incubation time was strain and drug specific. RPMI 1640 was the test medium throughout, with the exception of the use of YNB for Cryptococcus. Inhibition endpoints for compound 1A were recorded at 50% and 100% after both 24 h and 48 h incubation; caspofungin results against Aspergillus strains were read as minimum effective concentrations (MEC), the lowest concentration that leads to small, rounded, compact growth as compared to the confluent growth of the control.

Minimum Fungicidal Concentration: MFC determinations were performed according to the modifications previously described by Canton et al., Diagn. Microbiol. Infect. Dis., 45:203-206, 2003, and Ghannoum and Isham, Infectious Diseases in Clinical Practice, 15(4):250-253, 2007.

Specifically, the total contents of each clear well from the MIC assay were subcultured onto potato dextrose agar. To avoid antifungal carryover, the aliquots were allowed to soak into the agar and then were streaked for isolation once dry, thus removing the cells from the drug source. Fungicidal activity is defined as ≥99.9% reduction in the number of colony forming units (CFU)/ml from the starting inoculum count, while fungistatic activity is defined as <99.9% reduction. A drug is considered to be fungicidal if its MFC/MIC ratio is ≤4 or fungistatic if the ratio is >4. MIC and MFC values are considered to be equivalent if they are within 2 dilutions.

Determination of End Points: All compound 1A MIC endpoints were recorded at both 50% and 100% inhibition as compared to the growth controls. Though compound 1A did show both 50% and 100% inhibition against all Paecilomyces spp. and C. parapsilosis strains, all MIC values are hereafter reported at the 100% inhibition endpoint.

Determination of Incubation Times: Inhibition of growth following exposure to compound 1A was recorded after 24 and 48 hours of incubation. Exceptions to this were the mucormycetes, which were read only at 24 h because of their rapid growth rate, and the Cryptococcus strains, which were read after 72 h to coincide with the incubation period for all other comparators. There were no differences noted between the MIC values recorded at 24 h and at 48 h in any of the species tested. However, since some strains of Penicillium spp. and Paecilomyces spp. showed no visible growth in the MIC assay at 24 hrs, all MIC values are hereafter reported at the 48 h incubation time point.

Results

MIC and MFC Determinations Against Candida Strains: Table 5 shows the MIC and MFC data of compound 1A and comparators against C. albicans, including both fluconazole-susceptible (n=13) and resistant strains (n=7). MIC₅₀ is defined as the lowest concentration to inhibit 50% of the strains tested and MIC₉₀ is defined as the lowest concentration to inhibit 90% of the strains tested. MFC₅₀ is defined as the lowest concentration to kill 50% of the strains tested and MFC₉₀ is defined as the lowest concentration to kill 90% of the strains tested.

TABLE 5

| | | | | | | | | | |
|---|---|---|---|---|---|---|---|---|---|
| 1A | 1A | AmB | AmB | Cas | Cas | Flu | Flu | Vor | Vor |
| MIC | MFC | MIC | MFC | MIC | MFC | MIC | MFC | MIC | MFC |

Fluconazole-resistant n = 7

| | | | | | | | | | | |
|---|---|---|---|---|---|---|---|---|---|---|
| Range | 1-2 | 1->4 | 0.25-1 | 0.5-1 | 0.125-2 | 0.5->32 | 32->32 | >32 | <0.06->32 | 16->32 |
| MIC/MFC₅₀ | N/A | N/A | N/A | N/A | N/A | N/A | N/A | N/A | N/A | N/A |
| MIC/MFC₉₀ | N/A | N/A | N/A | N/A | N/A | N/A | N/A | N/A | N/A | N/A |

Fluconazole-susceptible n = 13

| | | | | | | | | | | |
|---|---|---|---|---|---|---|---|---|---|---|
| Range | 0.125-1 | 0.125-1 | 0.125-0.5 | 0.125-1 | 0.125-0.25 | 0.5-32 | 0.125-0.5 | 8->32 | <0.06-0.06 | >32 |
| MIC/MFC₅₀ | 0.5 | 0.5 | 0.25 | 0.25 | 0.25 | 0.25 | 0.25 | >32 | 0.6 | >32 |
| MIC/MFC₉₀ | 1 | 1 | 0.5 | 1 | 0.25 | 2 | 0.25 | >32 | 0.06 | >32 |

Table 6 shows fungicidal activity against C. glabrata strains for compound 1A and comparators.

TABLE 6

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 0.25-2 | 0.5-2 | 0.125-1 | 0.5-1 | 0.25-8 | 1->32 | 2->64 | >64 | 0.06->32 | >32 |
| MIC/MFC₅₀ | 1 | 1 | 0.5 | 0.5 | 0.5 | 1 | 8 | >64 | 0.25 | >32 |
| MIC/MFC₉₀ | 2 | 2 | 0.5 | 1 | 2 | 32 | >64 | >64 | 4 | >32 |

Table 7 shows the MIC and MFC data for all drugs against C. krusei strains.

TABLE 7

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 0.5-2 | 0.5-2 | 0.5-1 | 1 | 0.5-16 | 2->32 | 2-32 | >64 | <0.06-0.5 | 2->8 |
| MIC/MFC₅₀ | 1 | 1 | 0.5 | 1 | 1 | 2 | 16 | >64 | 0.125 | >8 |
| MIC/MFC₉₀ | 2 | 2 | 1 | 1 | 4 | 8 | 32 | >64 | 0.25 | >8 |

Table 8 shows fungicidal activity against the C. parapsilosis.

TABLE 8

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 0.25-2 | 0.25-2 | 0.125-1 | 0.5-2 | 0.25-2 | 2->32 | 0.25->64 | >64 | <0.06->32 | 1->32 |
| MIC/MFC₅₀ | 0.25 | 0.5 | 0.25 | 1 | 0.5 | >32 | 0.5 | >64 | <0.06 | >32 |
| MIC/MFC₉₀ | 2 | 2 | 0.5 | 1 | 2 | >32 | 0.5 | >64 | 0.06 | >32 |

Table 9 shows fungicidal activity against C. tropicalis for compound 1A and the comparators.

TABLE 9

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 0.5-8 | 0.5-16 | 0.25-0.5 | 0.25-1 | 0.06-2 | 0.5->32 | 0.25-2 | >64 | <0.06-1 | >32 |
| MIC/MFC₅₀ | 1 | 1 | 0.25 | 0.5 | 0.25 | 32 | 0.25 | >64 | 0.06 | >32 |
| MIC/MFC₉₀ | 8 | 8 | 0.5 | 1 | 0.5 | >32 | 0.5 | >64 | 0.06 | >32 |

Table 10 summarizes the MIC and MFC data for compound 1A and comparators against all Candida strains.

TABLE 10

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 0.125-8 | 0.125-16 | 0.125-1 | 0.125-2 | 0.06-16 | 0.5->32 | 0.125->32 | 8->32 | <0.06->32 | 1->32 |
| MIC/MFC₅₀ | 1 | 1 | 0.5 | 0.5 | 0.5 | 16 | 0.5 | >32 | 0.06 | >32 |
| MIC/MFC₉₀ | 2 | 2 | 0.5 | 1 | 2 | >32 | >32 | >32 | 1 | >32 |

MIC and MFC Determinations Against Cryptococcus Strains

Table 11 demonstrates activity against the Cr. neoformans strains for compound 1A and its comparators.

TABLE 11

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 0.5-2 | 0.5->4 | 0.06-1 | 0.25-2 | 1-16 | 16->32 | <0.06-16 | 2 | <0.06-0.25 | 0.06->0.5 |
| MIC/MFC₅₀ | 1 | 1 | 0.25 | 0.5 | 16 | >32 | 2 | >64 | <0.06 | >0.5 |
| MIC/MFC₉₀ | 1 | >4 | 0.5 | 1 | 16 | >32 | 8 | >64 | 0.125 | >0.5 |

MIC and MFC Determinations Against Aspergillus Strains

Tables 12-15 show the MIC and MFC data for compound 1A and comparators against the individual Aspergillus spp. Table 16 is a summary of the MIC and MFC data for all Aspergillus strains tested.

TABLE 12

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 1-4 | 2->4 | 1-8 | 1->32 | 0.125-8 | 8->32 | 32->64 | >64 | 0.25-4 | 1->16 |
| MIC/MFC₅₀ | 2 | 4 | 8 | 8 | 0.25 | >32 | >64 | >64 | 1 | 4 |
| MIC/MFC₉₀ | 2 | >4 | 8 | >32 | 0.5 | >32 | >64 | >64 | 2 | >16 |

TABLE 13

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 1-2 | 1->4 | 1-2 | 1->8 | 0.25-1 | 32->32 | 64 | 64 | 0.25-2 | 1->8 |
| MIC/MFC₅₀ | 1 | 2 | 1 | >8 | 0.25 | >32 | 64 | 64 | 0.5 | 8 |
| MIC/MFC₉₀ | 2 | >4 | 2 | >8 | 0.5 | >32 | 64 | 64 | 1 | >8 |

TABLE 14

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 1-2 | 2->4 | 0.25-2 | 0.25->8 | 0.125-2 | 16->32 | 32->64 | >64 | 0.06-8 | 0.5->8 |
| MIC/MFC₅₀ | 2 | 2 | 1 | 2 | 0.125 | >32 | >64 | >64 | 1 | >8 |
| MIC/MFC₉₀ | 2 | 4 | 1 | >8 | 0.25 | >32 | >64 | >64 | 4 | >8 |

TABLE 15

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 0.5-4 | 2->4 | 0.25-16 | 2->32 | 0.25-1 | 32->32 | 4->64 | >64 | 0.25-2 | 1->4 |
| MIC/MFC₅₀ | 2 | >4 | 8 | >32 | 0.25 | >32 | 64 | >64 | 0.5 | >4 |
| MIC/MFC₉₀ | 4 | >4 | 8 | >32 | 0.5 | >32 | >64 | >64 | 0.5 | >4 |

TABLE 16

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 0.5-4 | 1->4 | 0.25-16 | 0.25->8 | 0.125-8 | 8->32 | 4->64 | >64 | 0.06-8 | 0.5->8 |
| MIC/MFC₅₀ | 2 | 4 | 1 | >8 | 0.25 | >32 | >64 | >64 | 0.5 | >8 |
| MIC/MFC₉₀ | 2 | >4 | 8 | >8 | 0.5 | >32 | >64 | >64 | 2 | >8 |

MIC and MFC Determinations Against Difficult to Treat/Rare Fungi

Table 17 shows the MIC and MFC data for the Fusarium strains.

TABLE 17

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 1-4 | 1-4 | 0.5-2 | 1-4 | 1-64 | >32 | 8->64 | >64 | 2->32 | 8->32 |
| MIC/MFC₅₀ | 2 | 2 | 1 | 2 | 32 | >32 | >64 | >64 | 16 | 32 |
| MIC/MFC₉₀ | 4 | >4 | 2 | 2 | 32 | >32 | >64 | >64 | >32 | >32 |

Table 18 shows the MIC and MFC data for the Fusarium strains.

TABLE 18

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 1-4 | 1->4 | 0.25-2 | 0.5->8 | 32->32 | >32 | >32 | >32 | 8->16 | 16->16 |
| MIC/MFC₅₀ | 2 | 2 | 0.5 | 1 | >32 | >32 | >32 | >32 | 16 | >16 |
| MIC/MFC₉₀ | 4 | >4 | 2 | 8 | >32 | >32 | >32 | >32 | >16 | >16 |

Results against Penicillium and Paecilomyces strains can be found in Tables 19 and 20, respectively. Table 20 contains only concentration ranges, as the number of Paecilomyces strains (5) was too few to calculate the MIC₅₀ and MIC₉₀ values.

TABLE 19

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 0.5-2 | 2->4 | 0.5-4 | 1->8 | 0.25->32 | 16->32 | 1->64 | 64->64 | 0.5->32 | 1->32 |
| MIC/MFC₅₀ | 2 | 2 | 1 | 4 | 4 | >32 | >64 | >64 | 4 | >32 |
| MIC/MFC₉₀ | 2 | 4 | 2 | >8 | 32 | >32 | >64 | >64 | >32 | >32 |

TABLE 20

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | 0.5->4 | 4->4 | 0.25->8 | >4 | 1->32 | 32->32 | 32->64 | >64 | 0.125-2 | 1->8 |
| MIC/MFC₅₀ | N/A | N/A | N/A | N/A | N/A | N/A | N/A | N/A | N/A | N/A |
| MIC/MFC₉₀ | N/A | N/A | N/A | N/A | N/A | N/A | N/A | N/A | N/A | N/A |

MIC and MFC Determinations Against Dimorphic Fungi

Table 21 shows the MIC data for the Blastomyces dermatitidis, Coccidioides immitis, and Histoplasma capsulatum strains. MFCs were not performed on these fungi as these are restricted fungi against which there is no standardized method for performing MFCs.

TABLE 21

| | 1A | 1A | AmB | AmB | Cas | Cas | Flu | Flu | Vor | Vor |
|---|---|---|---|---|---|---|---|---|---|---|
| | MIC | MFC | MIC | MFC | MIC | MFC | MIC | MFC | MIC | MFC |
| Range | <0.03-0.25 | ND | <0.03-0.125 | ND | 0.03-0.5 | ND | 2-32 | ND | <0.03-0.125 | ND |

ND = not determined due to lack of a standardized method

MIC and MFC Determinations Against Other Candida Strains with Elevated MICs to Other Antifungals

Table 22 compares the MIC data of compound 1A against Candida strains with elevated MICs to the non-polyene comparators (n=17).

TABLE 22

| | 1A | 1A | Cas | Cas | Flu | Flu | Vor | Vor |
|---|---|---|---|---|---|---|---|---|
| Species | MIC | MFC | MIC | MFC | MIC | MFC | MIC | MFC |
| Albicans | 2 | 2 | | | >64 | >64 | 32 | >32 |
| Albicans | 1 | 1 | | | >32 | >32 | | |
| Albicans | 1 | 1 | | | >32 | >32 | | |
| Albicans | 1 | 1 | | | 32 | >32 | | |
| Albicans | 1 | 1 | 1 | 32 | >32 | >32 | | |
| Albicans | 1 | 1 | 1 | >32 | 32 | >32 | >0.06 | 16 |
| Albicans | 1 | >4 | 2 | >32 | >32 | >32 | 0.5 | >32 |
| Glabrata | 1 | 1 | 2 | 32 | 32 | >64 | 1 | >32 |
| Glabrata | 1 | 1 | 1 | 32 | 2 | >64 | >32 | >32 |
| Glabrata | 1 | 1 | | | >64 | >64 | 1 | >32 |
| Glabrata | 1 | 1 | 2 | 16 | >64 | >64 | 1 | >32 |
| Glabrata | 1 | 1 | 4 | >32 | 8 | >64 | 0.25 | >32 |
| Glabrata | 2 | 2 | 8 | 32 | 64 | >64 | 2 | >32 |
| Krusei | 0.5 | 2 | 16 | >32 | 16 | >64 | | |
| Parapsilosis | 0.5 | 0.5 | 2 | >32 | 0.25 | >64 | 0.06 | >32 |
| Parapsilosis | 0.5 | 0.5 | 2 | >32 | 0.5 | >64 | 0.06 | >32 |
| Parapsilosis | 1 | 1 | 2 | >32 | 1 | >64 | >32 | >32 |

### Example 17

The efficacy of compound 1A was tested and compared to AmBisome, voriconazole, and caspofungin in the treatment of disseminated Aspergillosis in an immunocompromised murine model.

Female CD-1 mice (Charles River Laboratories, Wilmington, Mass.) of about 30 g each were used as the model. Environmental controls for the animal room were set to maintain a temperature of 16 to 22° C., a relative humidity of 30-70% and a 12:12 light-dark cycle.

Preparation of Standard Inoculum

Organism: Aspergillus fumigatus AF91 was obtained from the Culture Collection of CWRU Center for Medical Mycology. From the frozen stock, the cells were sub-cultured in Potato Dextrose Agar (PDA) plates. Cells were then harvested using sterile saline with 0.05% Tween 80, centrifuged, and washed three times with normal saline (0.85% NaCl). A challenge inoculum of 1×10⁷ was prepared using a hemacytometer.

Verification of Inoculum Count: To check the inoculum count, ten-fold dilutions of A. fumigatus working conidial suspension was plated onto PDA media. The plates were incubated at 37° C. for 2-4 days and the colony counts determined.

Immunosuppression: Mice received subcutaneous cyclophosphamide in the following doses: 150 mg/kg, 4 days before infection, 100 mg/kg 1 day before infection, and 100 mg/kg 2 days after inoculation. On the day of the challenge, blood was collected from one mouse from each group for a white blood cell count to verify immunosuppression.

Infection: Each mouse was challenged with 1×10⁷ conidia in 0.1 ml of normal saline (via the tail vein). Animals were considered infected after successful IV dosing of the inoculum and inoculum confirmation (see section 7d). The efficacy of the treatment and control groups was assessed using tissue fungal burden and survival as indicators, using 5 mice per group (selected randomly) for tissue fungal burden and 10 mice per group for survival.

Test Compounds: The sponsor provided the test article, compound 1A (batch ELN EXP-11-AJ1675, potency 928 mg/g). It was administered intravenously in a solution of 5% aqueous glucose containing 5% dimethylsulfoxide (DMSO) prepared on the day of use from stock solutions of compound 1A in DMSO stored at −20° C. The comparators (AmBisome, voriconazole and caspofungin) were purchased by the Center for Medical Mycology from the pharmacy. They were dissolved in sterile water according to manufacturer's instructions and aliquots containing the selected doses taken.

In this report doses and concentrations of AmBisome are expressed as content of Amphotericin B.

Treatment Groups: Infected mice were randomized into the following groups (5 for tissue fungal burden, and 10 for survival per group).

Experiment I—Treatment groups were as follows: compound 1A 1.0 mg/kg, compound 1A 0.5 mg/kg, AmBisome 7.5 mg/kg, AmBisome 3.5 mg/kg, voriconazole 7.5 mg/kg, caspofungin 1.0 mg/kg, vehicle (5% aqueous glucose containing 5% DMSO), and an untreated control group.

Experiment II—Treatment groups were as follows: compound 1A 1.0 mg/kg, compound 1A 0.5 mg/kg, AmBisome 1.0 mg/kg, AmBisome 0.5 mg/kg, and an untreated control group. All treatments were given intravenously.

Schedule of Treatment: Beginning two hours post inoculation, animals were treated for a period of seven days. compound 1A and caspofungin were given once a day, AmBisome once every other day in Experiment I and every day in Experiment II. Voriconazole due to its rapid clearance, was given twice a day, 8 h apart.

Tissue Fungal Burden: Mice were sacrificed one day after the last day of treatment; then kidneys, and lungs were removed aseptically and weighed. Tissues were homogenized and serially diluted in Phosphate Buffered Saline. The homogenates were cultured for 48 h on PDA plates to determine the colony forming units (CFU); tissue fungal burdens were expressed as CFUs/gram of tissue.

Survival Analysis: Infected mice were monitored and any signs of illness (i.e., lethargy, weight loss, general failure to thrive), or mortality was recorded twice daily for up to 28 days post inoculation. An average weight for each treatment group was also recorded daily. Moribund animals that fail to take food/drink were euthanized.

Statistical Analysis: Differences in survival were compared using the Kaplan-Meier and mean CFUs in kidneys or lungs were compared using a nonparametric independent Mann-Whitney statistical test. A P-value of <0.05 was considered statistically significant.

Results

In vitro activity: Table 23 shows the in vitro activities of compound 1A and the comparative agents and of Amphotericin B against A. fumigatus AF9 1 (the infecting strain).

TABLE 23

| | | | Amphotericin | | |
|---|---|---|---|---|---|
| | 1A | AmBisome | B | Voriconazole | Caspofungin |
| MIC | 2 | 4 | 2 | 1 | 0.25 (MEC) |
| (μg/mL) | | | | | |
| MFC | 2 | N/A | 16 | >8 | >16 |
| (μg/mL) | | | | | |

Experiment I

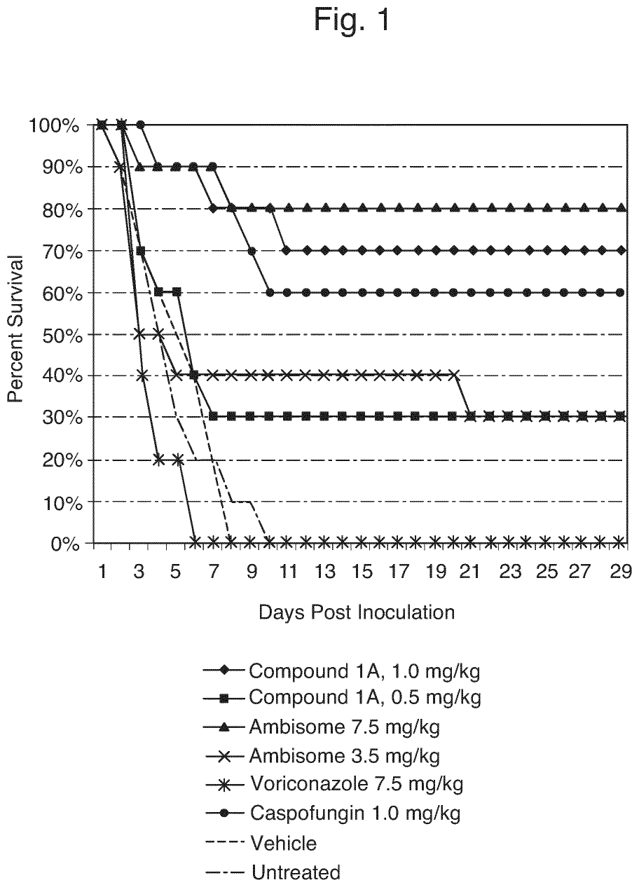

Survival: In FIG. 1, survival is given as a percentage of the total number of animals in a group on the first day of treatment.

Kidney Tissue Fungal Burden: Tissue fungal burden was assessed one day after the last treatment, or in the case of moribund animals, immediately after death (Table 24). The fungal burden of kidneys was analyzed and given as mean log CFUs±the standard deviation.

Lung Tissue Fungal Burden: Tissue fungal burden was assessed one day after the last treatment, or in the case of moribund animals, immediately after death (Table 24). The fungal burden of lungs was analyzed and given as mean log CFUs±the standard deviation.

TABLE 24

| | Mean log CFUs ± the standard deviation | |
|---|---|---|
| Treatments | Kidney | Lung |
| 1A 0.5 mg/kg | 3.16 ± 0.40 | 3.46 ± 0.49 |
| 1A 1.0 mg/kg | 0.51 ± 1.02 | 0.55 ± 1.10 |
| AmBisome 3.5 mg/kg | 0.45 ± 1.00 | 0.47 ± 1.06 |
| AmBisome 7.5 mg/kg | 0.98 ± 1.34 | 1.11 ± 1.52 |
| Voriconazole 7.5 mg/kg | 3.19 ± 0.59 | 3.38 ± 0.45 |
| Caspofungin 1.0 mg/kg | 3.11 ± 0.62 | 3.41 ± 0.64 |
| Vehicle control | 3.40 ± 0.18 | 3.56 ± 0.19 |
| Untreated contol | 3.34 ± 0.39 | 3.52 ± 0.20 |

Experiment II

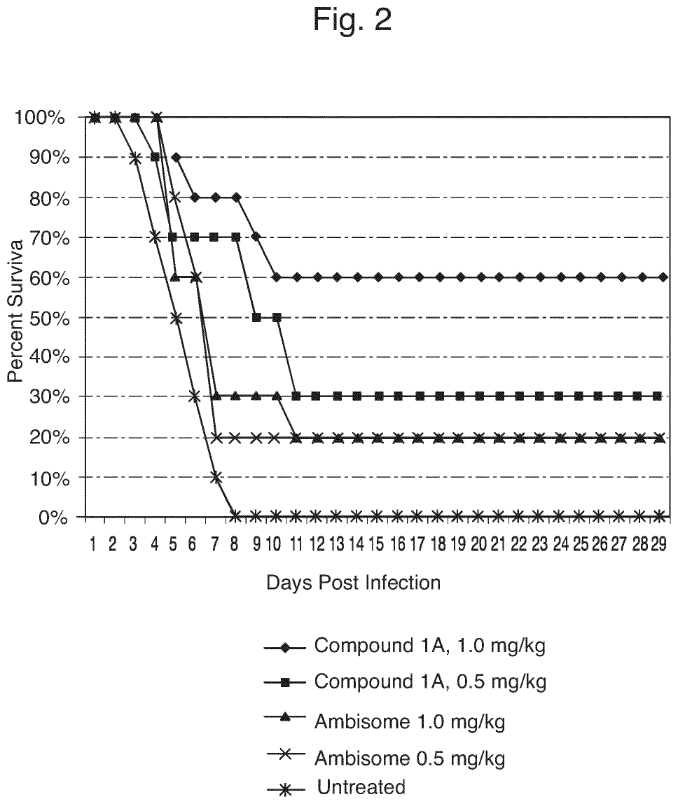

Survival: As can be seen in FIG. 2, survival was given as a percentage relative to the total number of animals in a group on the first day of treatment.

Kidney Tissue Fungal Burden: Tissue fungal burden was assessed one day after the last treatment, or in the case of moribund animals, immediately after death (Table 25). The fungal burden of kidneys was analyzed and given as mean log CFUs±the standard deviation.

Lung Tissue Fungal Burden: Tissue fungal burden was assessed one day after the last treatment, or in the case of moribund animals, immediately after death (Table 25). The fungal burden of lungs were analyzed and given as mean log CFUs±the standard deviation.

TABLE 25

| | Mean log CFUs ± the standard deviation | |
|---|---|---|
| Treatments | Kidney | Lung |
| 1A 0.5 mg/kg | 2.93 ± 0.73 | 2.75 ± 0.93 |
| 1A 1.0 mg/kg | 1.16 ± 1.10* | 1.17 ± 1.15* |
| AmBisome 0.5 mg/kg | 3.13 ± 0.94 | 2.37 ± 0.17 |
| AmBisome 1.0 mg/kg | 2.72 ± 1.06 | 2.26 ± 0.34 |
| Untreated control | 3.31 ± 0.37 | 3.30 ± 0.25 |

*P-value of <0.05 when compared to the untreated control group, the AmBisome 0.5 mg/kg-treated group and the 1A 0.5 mg/kg-treated group

### Example 18

Evaluating the efficacy compound 1A compared to AmBisome, voriconazole, fluconazole and caspofungin in the treatment of disseminated candidiasis in an immunocompromised murine model.

Female BALB/c mice (Charles River Laboratories, Wilmington, Mass.) of about 20 g each were used as the model. Environmental controls for the animal room were set to maintain a temperature of 16 to 22° C., a relative humidity of 30-70% and a 12:12 light-dark cycle.

Preparation of Standard Inoculum Organism: The clinical Candida albicans SC5314 strain was obtained from CMM Culture Collection and used as the infecting fungus. C. albicans was plated on Sabouraud Dextrose Agar (SDA) and incubated at 37° C. for 2 days. C. albicans cells were harvested by centrifugation and normal saline (0.85% NaCl) washes. A challenge inoculum of 5×10⁵ was prepared using a hemacytometer.

Verification of Inoculum Count: To check the inoculum count, ten-fold dilutions of C. albicans working conidial suspension was plated onto SDA media. The plates were incubated at 37° C. for 2 days and the colony counts determined.

Immunosuppression: Mice received subcutaneous cyclophosphamide in the following doses: 150 mg/kg, 4 days before infection, 100 mg/kg 1 day before infection, and 100 mg/kg 2 days after inoculation. On the day of the challenge, blood was collected from one mouse from each group for a white blood cell count to verify immunosuppression.

Infection: Each mouse was challenged with 1×10⁴ blastospores in 0.1 ml of normal saline (via the tail vein). Animals were considered infected after successful IV dosing of the inoculum and inoculum confirmation. The efficacy of the treatment and control groups was assessed using tissue fungal burden and survival as indicators, using 5 mice per group for tissue fungal burden and 10 mice per group for survival. The tissue burden and survival arms of Experiment I were performed separately on two different occasions.

Test Compounds: The sponsor provided the test article, compound 1A (batch ELN Exp-11-AJ1675, potency 928 mg/g). It was administered intravenously in a solution of 5% aqueous glucose containing 5% dimethylsulfoxide (DMSO) prepared on the day of use from stock solutions of compound 1A in DMSO stored at −20° C. The comparators (AmBisome, voriconazole, caspofungin and fluconazole) were purchased by the Center for Medical Mycology from the pharmacy. They were dissolved in sterile water according to manufacturer's instructions and aliquots containing the selected doses taken.

In this report doses and concentrations of AmBisome are expressed as content of Amphotericin B.

Treatment Groups: Infected mice were randomized into the following groups (5 for tissue fungal burden, and 10 for survival per group.

Experiment I—Treatment groups were as follows: compound 1A 0.7 mg/kg, compound 1A 0.35 mg/kg, AmBisome 5.4 mg/kg, AmBisome 2.7 mg/kg, voriconazole 4 mg/kg, caspofungin 0.35 mg/kg, fluconazole 6 mg/kg, vehicle (5% aqueous glucose containing 5% DMSO) and an untreated control group.

Experiment II—Treatment groups were as follows: compound 1A 0.7 mg/kg, compound 1A 0.35 mg/kg, AmBisome 0.7 mg/kg, AmBisome 0.35 mg/kg, and an untreated control group. All treatments were given intravenously.

Schedule of Treatment: Beginning two hours post inoculation, animals were treated for a period of seven days. With the exception of voriconazole, treatments were given once a day. Due to rapid clearance, the voriconazole was given twice a day, 8 hours apart.

Tissue Fungal Burden: Mice were sacrificed one day after the last day of treatment, kidneys, and brains were removed aseptically and weighed. Tissues were homogenized and serially diluted in Phosphate Buffered Saline. The homogenates were cultured for 48 hr on SDA plates to determine the colony forming units (CFU); tissue fungal burden was expressed as CFUs/gram of tissue.

Survival Analysis: Infected mice were monitored and any signs of illness (i.e. lethargy, weight loss, general failure to thrive), or mortality was recorded twice daily for up to 28 days post inoculation. An average weight for each treatment group was also recorded daily. Moribund animals that fail to take food/drink were euthanized.

Statistical Analysis: Differences in mean log CFU s in the kidneys or brain were compared using a nonparametric independent Mann-Whitney test. Differences in survival were compared using the Kaplan-Meier test. AP-value of <0.05 was considered statistically significant.

Results

In vitro activity: Table 26 shows the in vitro activities of compound 1A and the comparative agents and of Amphotericin B against C. albicans SC5314 (the infecting strain).

TABLE 26

| | | | Amphotericin | | | |
|---|---|---|---|---|---|---|
| | 1A | AmBisome | B | Voriconazole | Fluconazole | Caspofungin |
| MIC | 0.5 | 0.5 | 0.125 | 0.008 | 0.25 | 0.25 (MEC) |
| (μg/mL) | | | | | | |
| MFC | 0.5 | N/A | 0.25 | >32 | >64 | 32 |
| (μg/mL) | | | | | | |

Experiment I

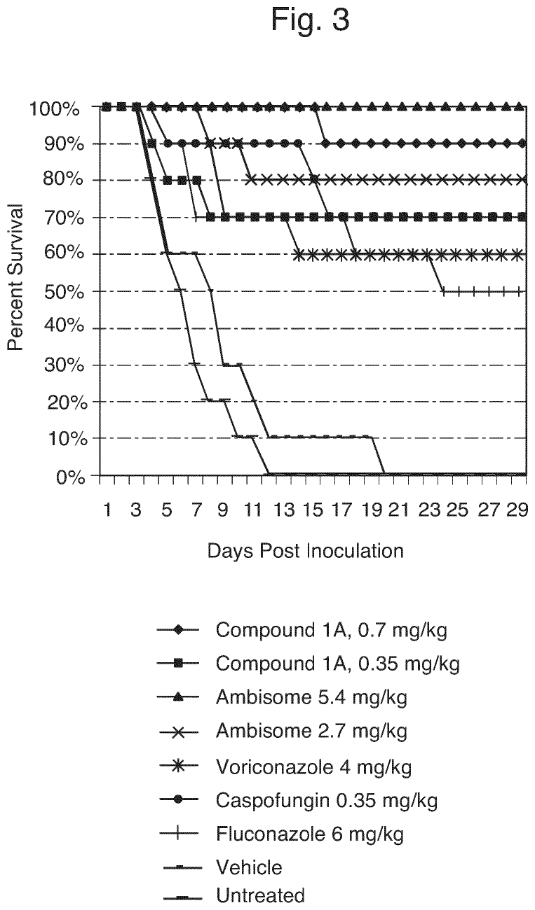

Survival: In FIG. 3, survival is given as a percentage of the total number of animals in a group on the first day of treatment.

Kidney Tissue Fungal Burden: Tissue fungal burden was assessed one day after the last treatment, or in the case of moribund animals, immediately after death (Table 27). The fungal burden of kidneys were analyzed and given as mean log CFUs±the standard deviation.

Brain Tissue Fungal Burden: Tissue fungal burden was assessed one day after the last treatment, or in the case of moribund animals, immediately after death (Table 27). The fungal burden of brains was analyzed and given as mean log CFUs±the standard deviation.

TABLE 27

| | Mean log CFUs ± the standard deviation | |
|---|---|---|
| Treatments | Kidney | Brain |
| 1A 0.35 mg/kg | 3.16 ± 1.41 | 2.92 ± 1.21 |
| 1A 0.7 mg/kg | 2.66 ± 2.11 | 3.59 ± 1.20 |
| AmBisome 2.7 mg/kg | 0.59 ± 0.93 | 0.14 ± 0.31 |
| AmBisome 5.4 mg/kg | 0.51 ± 2.76 | 2.01 ± 0.82 |
| Voriconazole 4 mg/kg | 3.45 ± 1.15 | 3.42 ± 2.30 |
| Caspofungin 0.35 mg/kg | 3.22 ± 1.26 | 1.22 ± 1.87 |
| Fluconazole 6 mg/kg | 3.87 ± 1.53 | 1.28 ± 1.95 |
| Vehicle control | 4.46 ± 0.56 | 3.39 ± 2.31 |
| Untreated control | 4.62 ± 0.59 | 3.48 ± 1.36 |

Experiment II

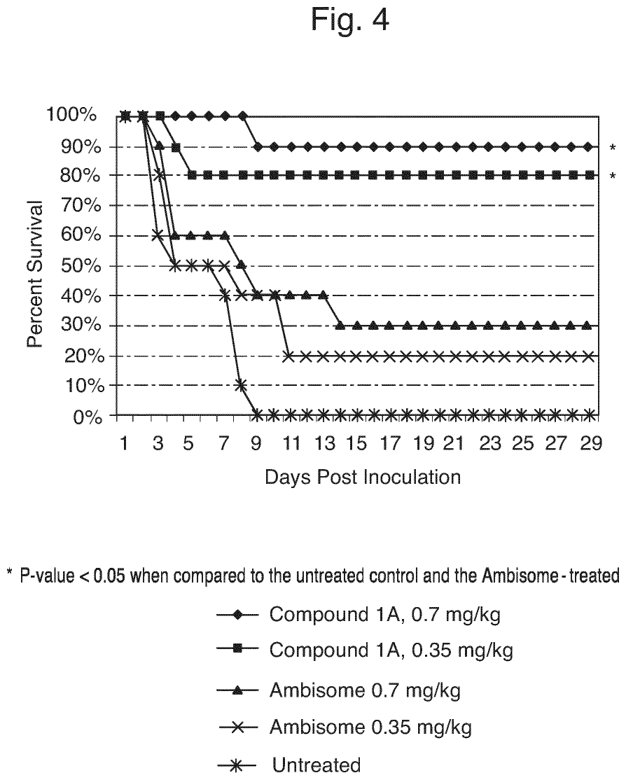

Survival: In FIG. 4, survival is given as a percentage relative to the total number of animals in a group on the first day of treatment.

Kidney Tissue Fungal Burden: Tissue fungal burden was assessed one day after the last treatment, or in the case of moribund animals, immediately after death (Table 28). The fungal burden of kidneys was analyzed and given as mean log CFUs±the standard deviation.

Brain Tissue Fungal Burden: Tissue fungal burden was assessed one day after the last treatment, or in the case of moribund animals, immediately after death (Table 28). The fungal burden of brains was analyzed and given as mean log CFUs±the standard deviation.

TABLE 28

| | Mean log CFUs ± the standard deviation | |
|---|---|---|
| Treatments | Kidney | Brain |
| 1A 0.35 mg/kg | 3.53 ± 0.63 | 3.55 ± 1.26 |
| 1A 0.7 mg/kg | 2.99 ± 1.05^{a,b} | 2.97 ± 2.04 |
| AmBisome 0.35 mg/kg | 4.33 ± 0.50 | 4.04 ± 0.31 |
| AmBisome 0.7 mg/kg | 4.27 ± 0.67 | 3.84 ± 0.68 |
| Untreated control | 4.57 ± 0.22 | 4.19 ± 0.84 |

^{a}P-value of <0.05 when compared to the untreated control group.
^{b}P-value of <0.05 when compared to the AmBisome 0.35-treated group

### Example 19

In total, 74 batches of particles have been produced; 26 of these batches have been produced using PACA and 48 using poly(lactic co-glycolic acid) (PLGA).

The particles quality has been assessed using the diameter, polydispersity index (PDI), and stability in suspension as criteria. In particular, the prepared particles were characterized as well-behaving, acceptable, and poorly behaving as follows:
well-behaving particle formulations were stable suspensions having a z-averaged diameter of <200 nm, PDI of <0.3, and no visible agglomerates;
acceptable particle formulations were stable suspensions with PDI of >0.31 and
poorly behaving particle formulations were unstable suspensions susceptible to phase separation and sedimentation.

The summary of the formulation produced in this example is provided in Table 29.

TABLE 29

| | | Formulation |
|---|---|---|
| # | Formulation | Properties |
| 1 | POPZ | well |
| | | behaving |
| 2 | PACA | poorly |
| | | behaving |
| 3 | PACA | acceptable |
| 4 | PACA | poorly |
| | | behaving |
| 5 | PACA | acceptable |
| 6 | PACA | acceptable |
| 7 | PACA | acceptable |
| 8 | PACA | acceptable |
| 9 | PACA | poorly |
| | | behaving |
| 10 | PEHCA | poorly |
| | | behaving |
| 11 | PEHCA | poorly |
| | | behaving |
| 12 | PEHCA | poorly |
| | | behaving |
| 13 | PEHCA | poorly |
| | | behaving |
| 14 | PEHCA pH1 | well |
| | | behaving |
| 15 | PEHCA pH2 | well |
| | | behaving |
| 16 | PEHCA pH3 | poorly |
| | | behaving |
| 17 | PEHCA pH4 | poorly |
| | | behaving |
| 18 | PEHCA pH4 | poorly |
| | | behaving |
| 19 | PEHCA, 5% | acceptable |
| | vanillin, pH2 | |
| 20 | PEHCA, 5% | acceptable |
| | vanillin, pH2, | |
| | acetone | |
| 21 | PEHCA, 5% | poorly |
| | vanillin, pH2, | behaving |
| | acetone | |
| 22 | PEHCA, 5% | acceptable |
| | vanillin, pH2 | |
| 23 | PEHCA, 5% | poorly |
| | vanillin, pH2, | behaving |
| | acetone | |
| 24 | PEHCA, 5% | poorly |
| | vanillin, pH2, | behaving |
| | acetone | |
| 25 | Compatibility study | |
| | lipids | |
| 26 | Test nanoassembly | poorly |
| | PLGA | behaving |
| 27 | Extended | |
| | compatibility study | |
| | lipids and oils | |
| 28 | PLGA particle | well |
| | synthesis | behaving |
| 29 | PLGA, PVA 2%, | acceptable |
| | NMP | |
| 30 | PLGA, Plu F68 1%, | poorly |
| | NMP | behaving |
| 31 | PLGA, PVA 2%, | poorly |
| | 10% NMP in acetone | behaving |
| 32 | PLGA, Plu F68 2%, | poorly |
| | 10% NMP in acetone | behaving |
| 33 | PLGA, Plu F68 1%, | poorly |
| | 10% NMP in acetone | behaving |
| 34 | PLGA | poorly |
| | | behaving |
| 35 | PLGA | poorly |
| | | behaving |
| 36 | PLGA | poorly |
| | | behaving |
| 37 | PLGA | poorly |
| | | behaving |
| 38 | PLGA | poorly |
| | | behaving |
| 39 | PLGA | poorly |
| | | behaving |
| 40 | PLGA | poorly |
| | | behaving |
| 41 | PLGA | poorly |
| | | behaving |
| 42 | PEHCA | acceptable |
| 43 | PEHCA | poorly |
| | | behaving |
| 44 | PEHCA | acceptable |
| 45 | PEHCA | acceptable |
| 46 | PLGA, Plu F68 2%, | poorly |
| | NMP | behaving |
| 47 | PLGA, Plu F127 2%, | well |
| | NMP | behaving |
| 48 | PLGA, Plu F127 4%, | well |
| | NMP | behaving |
| 49 | PLGA, Tween80 2%, | well |
| | NMP | behaving |
| 50 | PLGA, Plu F68 2%, | well |
| | 50% NMP in DMSO | behaving |
| | | fresh, poorly |
| | | behaving |
| | | after dialysis |
| 51 | PLGA, Plu F127 2%, | well |
| | 50% NMP in DMSO | behaving |
| 52 | PLGA, Plu F127 4%, | well |
| | 50% NMP in DMSO | behaving |
| 53 | PLGA, Tween80 2%, | well |
| | 50% NMP in DMSO | behaving |
| | | fresh, poorly |
| | | behaving |
| | | after dialysis |
| 54 | PLGA, Plu F127 4%, | well |
| | 50% NMP in DMSO | behaving |
| | | fresh, poorly |
| | | behaving |
| | | after dialysis |
| 55 | PEHCA polymerization | acceptable |
| | with 1% V65 and 20% | after |
| | kolliphor | centrifugation |
| 56 | PEHCA polymerization | acceptable |
| | with 5% V65 and 20% | after |
| | kolliphor | centrifugation |
| 57 | PEHCA polymerization | poorly |
| | with 5% V65 and 20% | behaving |
| | kolliphor and 5% vanillin | |
| 58 | PEHCA polymerization | well |
| | with 5% V65 and 20% | behaving |
| | kolliphor, 5% vanillin | |
| | and 10% Compound 1A | |
| 59 | PVA 2%, NMP, 5% | well |
| | Compound 1A, 5% vanillin | behaving |
| 60 | PVA 4%, NMP, 5% | poorly |
| | Compound 1A, 5% vanillin | behaving |
| 61 | F68 2%, NMP, 5% | poorly |
| | Compound 1A, 5% vanillin | behaving |
| 62 | PVA 2%, NMP, 10% | poorly |
| | Compound 1A | behaving |
| 63 | PVA 4%, NMP, 10% | poorly |
| | Compound 1A | behaving |
| 64 | F68 2%, NMP, 10% | poorly |
| | Compound 1A | behaving |
| 65 | PLGA, Plu F127 2%, | well |
| | Vanillin NMP | behaving |
| 66 | PLGA, Plu F127 4%, | well |
| | Vanillin NMP | behaving |
| 67 | Tween 80 2% | well |
| | Vanillin NMP | behaving |
| 68 | Tween 80 4% | well |
| | Vanillin NMP | behaving |
| 69 | PLGA, Plu F127 2%, | well |
| | NMP | behaving |
| 70 | PLGA, Plu F127 4%, | well |
| | NMP | behaving |
| 71 | Tween 80 2%, NMP | well |
| | | behaving |
| 72 | Tween 80 4%, NMP | well |
| | | behaving |
| 73 | PLGA, Plu F127 2%, | acceptable |
| | DMF | |
| 74 | PLGA, Plu F127 4%, | acceptable |
| | DMF | |
| 75 | Tween 80 2% DMF | acceptable |
| 76 | Tween 80 4% DMF | acceptable |
| 77 | PEHCA, vanillin, | well |
| | Compound 1A, | behaving |
| | Kolliphor | |
| 78 | PEHCA, 1,6-HBCA, | well |
| | vanillin, Compound 1A, | behaving |
| | Kolliphor | |
| 79 | PEHCA, vanillin, | well |
| | Compound 1A, | behaving |
| | Kolliphor, Brij | |
| 80 | PEHCA, 1,6-HBCA, | poorly |
| | vanillin, Compound 1A, | behaving |
| | Kolliphor | |
| 81 | PEHCA, vanillin, | poorly |
| | Compound 1A, | behaving |
| | Kolliphor, Brij | |
| 82 | PEHCA, vanillin, | poorly |
| | Compound 1A, | behaving |
| | Kolliphor, Brij | |
| 83 | PEHCA, thymoquinone, | poorly |
| | Compound 1A, | behaving |
| | Kolliphor, Brij | |
| 84 | PEHCA, thymoquinone, | poorly |
| | Compound 1A, | behaving |
| | Kolliphor, Brij | |
| 85 | Methanol/chloroform, | poorly |
| | Plu 2% | behaving |
| 86 | Methanol/chloroform, | poorly |
| | Plu 4% | behaving |
| 87 | Methanol/chloroform, | poorly |
| | Tween 80 2% | behaving |
| 88 | Methanol/chloroform, | poorly |
| | Tween 80 4% | behaving |

Formulation 45 was prepared using the following components.

| | Amount | Unit |
|---|---|---|
| Aqueous phase | | |
| Kolliphor HS 15 | 0.048 | g |
| Brij L23 | 0.048 | g |
| 10 mM HCl. pH 2 | 4.8 | g |
| 6-O-Palmitoyl-L-ascorbic acid | 0.0048 | |
| Organic phase | | |
| 2-ethylhexylcyanoacrylate | 0.32 | g |
| Compound 1A | 0.0384 | g |
| Vanillin | 0.04 | g |
| Miglyol 812 | 0.0056 | g |
| Total: | 5.30 | g |

Formulation 49 was prepared using the following components.

| | Amount | Unit |
|---|---|---|
| Organic phase | | |
| Compound 1A | 0.005 | g |
| Poly(Lactide-co-Glycolide) | 0.05 | g |
| A-Methyl-2-pyrrolidone | 2.5 | ml |
| Aqueous phase (pH 7) | | |
| Tween-80 | 0.05 | g |
| DI Water | 2.5 | ml |

Formulation 73 was prepared using the following components.

| | Amount | Unit |
|---|---|---|
| Organic phase | | |
| Compound 1A | 0.0025 | g |
| Poly(Lactide-co-Glycolide) | 0.05 | g |
| W-Dimethylformamide | 2.5 | ml |
| Aqueous phase (pH 7) | | |
| Pluronic F127 | 0.05 | g |
| DIW | 2.5 | ml |

Poly(Lactide-co-Glycolide) utilized in this example had lactide-glycolide ratio of 50:50, was ester terminated, and had a weight-averaged molecular weight of 38000-54000 Da.

PACA Particles

Multiple parameters for the production of PACA particles were varied, as described below.

Monomers: different types of PACA monomers were used for encapsulation of Compound 1A. The aim was to solubilize Compound 1A in the oil phase without starting a polymerization reaction. Poly(ethylhexyl cyanoacrylate) (PEHCA) was working together with Compound 1A. Poly(butyl cyanoacrylate) (PEBCA) was also tested. During these tests, it was found that Compound 1A initiates polymerization of PEBCA and that the resulting particles were unsatisfactory.

pH: Polymerization of particles at low pH (e.g., pH=1) produced particles with superior properties (size, size distribution and colloidal stability). Due to oxidation of Compound 1A at low pH, a higher pH of 4 was utilized. Retention of Compound 1A in the oil phase during the production was found to reduce its sensitivity to pH. Lower pH levels were tested to determine if particles with higher loading and loading efficiency. LC-DAD-QTOF analyses of these formulations demonstrated that no degradation took place, and pH is therefore not a critical parameter for Compound 1A stability.

Surfactants: Mixture of Brij L35 and Kolliphor HS15 were typically used throughout the tests described herein. Kolliphor was found to initiate the polymerization process.

Stabilizers: Ascorbic acid was tested for its effect on the stability of Compound 1A in solution. Ascorbic acid was added to the aqueous phase during polymerization. Compound 1A was found to be chemically dehydrated at high concentrations of ascorbic acid. Ascorbic acid was found to reduce stability of Compound 1A at high concentration.

Vanillin was tested for its effect on the stability of Compound 1A. A series of experiments was performed with and without vanillin. In these experiments, vanillin was found to assist in dissolution of Compound 1A. The addition of vanillin was also observed to reduce the stability of the Compound 1A/acrylate suspension. One possible explanation for this effect may be in the improved solubility of Compound 1A in the oil phase, as dissolved Compound 1A was observed to react more easily with the acrylate monomer compared to undissolved Compound 1A.

In General, the Constituents of a PACA Particle Contains:
Aqueous phase: Brij L35, Kolliphor HS15 and HCl to adjust pH
Oil phase: acrylate monomer (ethyl hexylcyanoacrylate, ethyl butylcyanoacrylate, 1-heptyl cyanoacrylate, and 2-phenylethyl cyanoacrylate/butyl cyanoacrylate), Miglyol, vanillin, Compound 1A. 1-Heptyl cyanoacrylate and 2-phenylethyl cyanoacrylate/butyl cyanoacrylate were unable to dissolve Compound 1A.

Key Findings:
PACA particles can stabilize Compound 1A (tested for 2 weeks) in suspension
Vanillin has a solubilizing effect on Compound 1A
PACA particles may be produced at low pH without causing unacceptable levels of Compound 1A decomposition
Ascorbic acid and 6-O-palmityl-L-ascorbic acid, water-soluble antioxidants, failed to improve the Compound 1A stability at the tested concentrations of ascorbic acid.

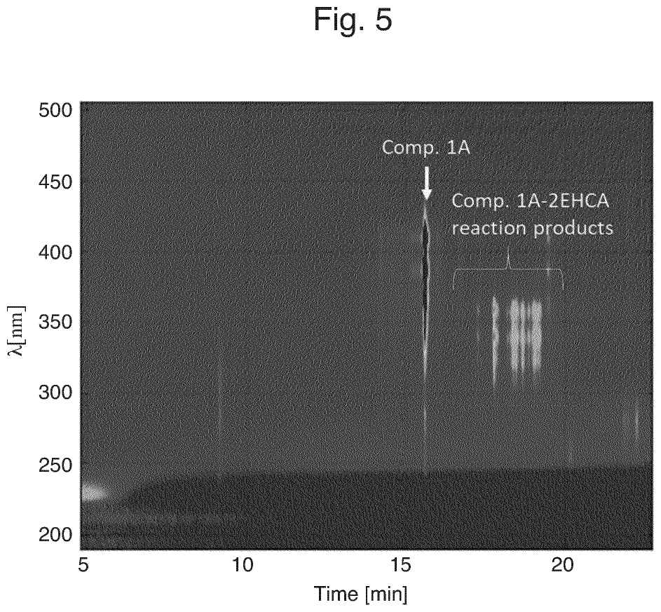

An LC-UV trace for one of the best-performing PACA formulation of Compound 1A is shown in FIG. 5.

PLGA Particles

PLGA nanoparticles have been investigated as a vehicle for Compound 1A. PLGA has certain advantageous properties over PACA. For example, the PLGA polymer is pre-formed. This means that the polymeric excipient does not involve reactivity-related issues. Additionally, PLGA allows the use of an organic solvent, thereby permitting full dissolution of Compound 1A before encapsulation.

The method utilized in the preparation of PLGA particles described herein was nanoprecipitation. This procedure involves a slow addition of the organic phase (containing PLGA, Compound 1A, and other, optional hydrophobic constituents, e.g., vanillin) to an aqueous solution containing a surfactant. Alternatively, microfluidics can be used, e.g., by mixing a fixed ratio of the organic phase and the aqueous phase in continuous flow through microfluidic channels.

During the initial testing of PLGA particles, the organic phase has been added to an aqueous solution with magnetically driven stirring. We have tested four different surfactants at 1-3 different concentrations. Additionally, we have tested a multitude of organic solvents and mixtures of organic solvents. The conditions tested are summarized in table 30.

Table 30 provides a summary of the conditions tested during PLGA production. The Compound 1A percentages represent theoretical maximum dry weight loading.

TABLE 30

| | | | NMP 5% | 10% NMP | 10% NMP | 10% NMP | 50% NMP | |
|---|---|---|---|---|---|---|---|---|
| | NMP | NMP | vanillin | in acetone | in ethanol | in DMSO | in DMSO | DMF |
| PVA 2% | Comp. 1A | — | Comp. 1A | Comp. 1A | Comp. 1A | Comp. 1A | Comp. 1A | — |
| | 10% ² | | 5% ¹ | 10% ³ | 10% ³ | 10% ³ | 10% ³ | |
| PVA 4% | Comp. 1A | — | Comp. 1A | — | — | — | — | — |
| | 10% ³ | | 5% ³ | | | | | |
| F68 1% | Comp. 1A | — | — | Comp. 1A | — | — | — | — |
| | 10% ³ | | | 10% ³ | | | | |
| F68 2% | Comp. 1A | — | Comp. 1A | Comp. 1A | Comp. 1A | Comp. 1A | Comp. 1A | — |
| | 10% ² | | 5% ³ | 10% ³ | 10% ³ | 10% ³ | 10% ³ | |
| F127 2% | Comp. 1A | Comp. 1A | Comp. 1A | — | Comp. 1A | Comp. 1A | Comp. 1A | Comp. 1A |
| | 10% ¹ | 5% ¹ | 5% ¹ | | 10% ³ | 10% ³ | 10% ³ | 5% ⁴ |
| F127 4% | Comp. 1A | Comp. 1A | Comp. 1A | — | — | — | — | Comp. 1A |
| | 10% ¹ | 5% ¹ | 5% ¹ | | | | | 5% ⁴ |
| Tween 80 2% | Comp. 1A | Comp. 1A | Comp. 1A | — | — | — | — | Comp. 1A |
| | 10% ¹ | 5% ¹ | 5% ¹ | | | | | 5% ⁴ |
| Tween 80 4% | Comp. 1A | Comp. 1A | Comp. 1A | — | — | — | — | Comp. 1A |
| | 5% ⁴ | 5% ¹ | 5% ¹ | | | | | 5% ⁴ |

¹ The formulation was a well-behaving formulation;
² The formulation was acceptable
³ The formulation was a poorly behaving formulation
⁴ No data are available for this formulation.

The current maximum load of Compound 1A achieved is 2.4%, however we wish to increase this to >5%.

In General, the PLGA Particles Include:
Aqueous phase: Poly(vinyl alcohol) (PVA), Pluronic F68 (F68), Pluronic F127 (F127), Tween 80. The concentration is given in (w/v) percentages.
Organic phase: Organic solvent, Compound 1A, and potentially other hydrophobic excipients (e.g. vanillin)

Key Findings:
NMP was the best performing solvent for the preparation of PLGA particles
Pluronic F127 and Tween 80 were some of the best-performing surfactants for the preparation of PLGA particles described herein.
Vanillin did not improve the drug loading of PLGA particles.
Compound 1A remains stable during the particle production.
The concentration of Compound 1A is low in the final suspension due to its dilution during production. Concentrations of Compound 1A may be increased by tangential flow filtration.

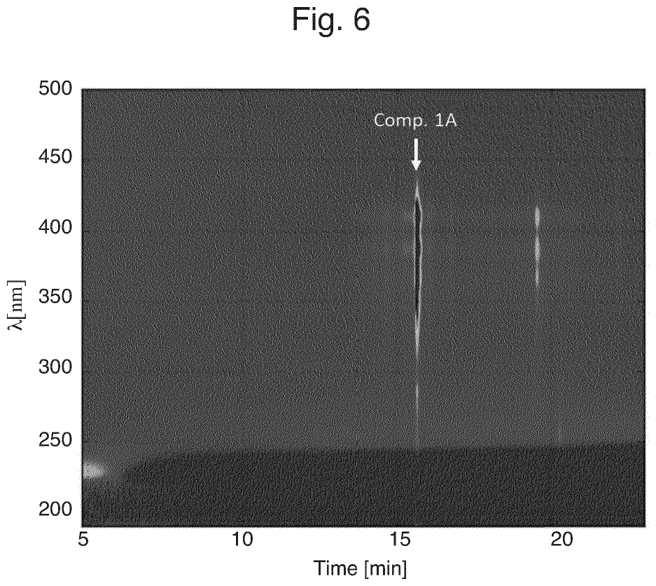

An LC-UV trace for one of the best-performing PLGA formulation of Compound 1A is shown in FIG. 6.

Lipid Particles

Lipid-based particles were investigated as a vehicle for Compound 1A. An investigation of Compound 1A solubility in different lipids and oils was performed as shown in the list below.
stearic acid
myristic acid
palmitic acid
isopropyl myristate
isopropyl palmitate
1-nonanol
linalool
eugenol
trans-cinnamaldehyde
linalyl acetate
p-anisaldehyde
tetraglycol

None of the tested oils or lipids dissolved Compound 1A well. Compound 1A was somewhat soluble only in trans-cinnamaldehyde.

### Example 20

Using the desolvation method, an organic solvent such as ethanol, acetone or N-methylpyrrolidone is slowly added to an aqueous solution of albumin. This caused the albumen to precipitate. The precipitated protein can be crosslinked using glutaraldehyde or transglutaminase. The protein particles are purified by centrifugation and resuspension followed by dialysis.

### Example 21

The albumin nanoparticles are produced by adding urea to an albumin solution. This destabilize the tertiary structure of the protein causing hydrophobic domains of the protein to be exposed to the aqueous phase, leading to particle formation. The precipitated protein can be crosslinked using glutaraldehyde or transglutaminase. The protein particles is purified by centrifugation and resuspension followed by dialysis.

### Example 22

The protein is destabilized as described in example 21 but at an elevated temperature instead. This also induced a certain amount of crosslinking of the proteins.

### Example 23

The same procedure was used for this example as that which is described in Examples 20-22 with the exception that the protein is fibroin.

### Example 24

The same procedure was used for this example as that which is described in Example 20-22 with the exception that the protein is gelatin.

### Example 25

To any of Examples 20-24, a compound 1A is swelled into the nanoparticles from a solution in N-methylpyrrolidone or DMSO.

### Other Embodiments

Various modifications and variations of the described invention will be apparent to those skilled in the art without departing from the scope and spirit of the invention. Although the invention has been described in connection with specific embodiments, it should be understood that the invention as claimed is not limited to such specific embodiments.

What is claimed is:

1. A pharmaceutical composition comprising a plurality of nanoparticles comprising an active pharmaceutical ingredient that is a compound of the following structure: 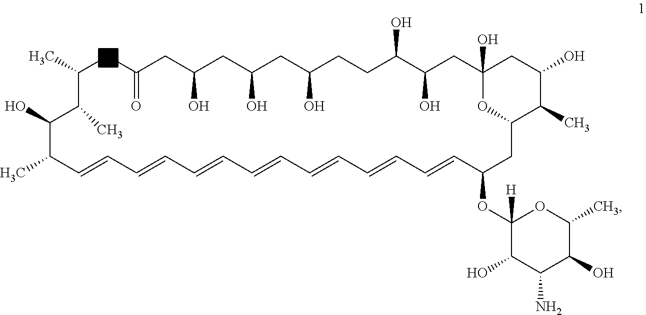 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)C[C@H]1C)C[C@H](O)[C@H]2C]
or a pharmaceutically acceptable salt thereof.

2. The pharmaceutical composition of claim 1, wherein the active pharmaceutical ingredient is 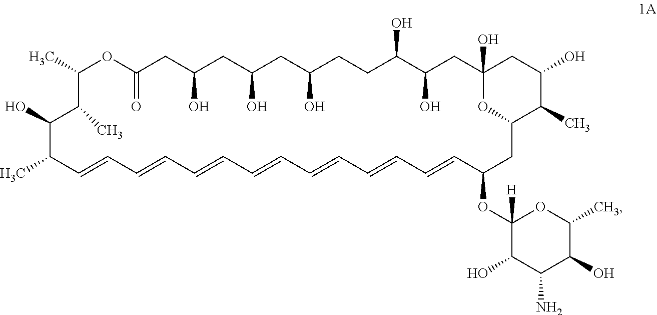 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C]
or a pharmaceutically acceptable salt thereof.

3. The pharmaceutical composition of claim 1 or 2, further comprising a pharmaceutically acceptable polymeric excipient.

4. The pharmaceutical composition of claim 3, wherein the plurality of nanoparticles comprises the pharmaceutically acceptable polymeric excipient.

5. The pharmaceutical composition of claim 4, wherein the active pharmaceutical ingredient is nanoencapsulated.

6. The pharmaceutical composition of any one of claims 3 to 5, wherein the pharmaceutically acceptable polymeric excipient is a poly(alkyl cyanoacrylate) or a polyphosphazene.

7. The pharmaceutical composition of claim 6, wherein the pharmaceutically acceptable polymeric excipient is a poly(alkyl cyanoacrylate).

8. The pharmaceutical composition of claim 7, wherein the pharmaceutically acceptable polymeric excipient is a poly(ethylhexyl cyanoacrylate), poly(ethyl cyanoacrylate), poly(n-hexyl cyanoacrylate), poly(4-methylpentyl cyanoacrylate), poly(ethylbutyl cyanoacrylate), poly(butyl cyanoacrylate), or poly(octyl cyanoacrylate).

9. The pharmaceutical composition of claim 8, wherein the pharmaceutically acceptable polymeric excipient is a poly(ethylhexyl cyanoacrylate).

10. The pharmaceutical composition of any one of claims 3 to 5, wherein the pharmaceutically acceptable polymeric excipient is poly(lactic-co-glycolic acid).

11. The pharmaceutical composition of any one of claims 3 to 5, wherein the pharmaceutically acceptable polymeric excipient is a protein.

12. The pharmaceutical composition of claim 11, wherein the pharmaceutically acceptable polymeric excipient is a protein that is casein, albumin, fibroin, gelatin, or a combination thereof.

13. A pharmaceutical composition comprising a plurality of nanoparticles comprising a poly(ethylhexyl cyanoacrylate) and an active pharmaceutical ingredient that is a compound of the following structure: 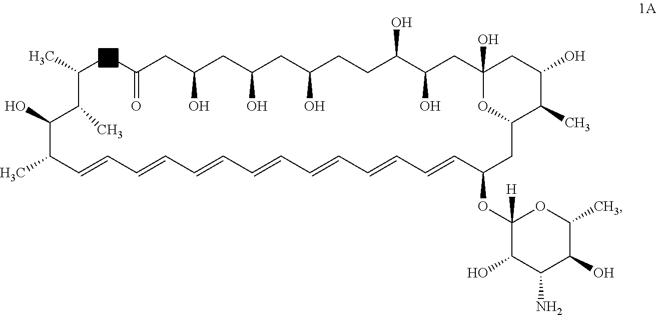 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)C[C@H]1C)C[C@H](O)[C@H]2C]
or a pharmaceutically acceptable salt thereof.

14. A pharmaceutical composition comprising a plurality of nanoparticles comprising a poly(lactic-co-glycolic acid) and an active pharmaceutical ingredient that is a compound of the following structure: 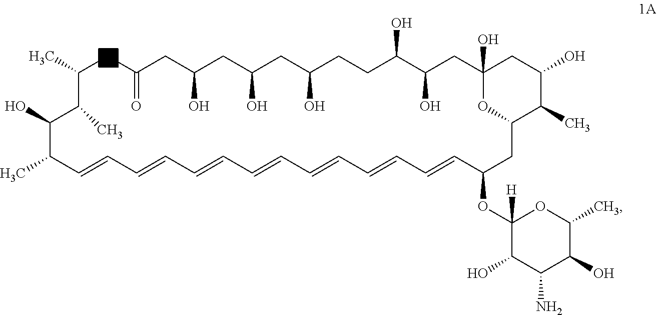 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)C[C@H]1C)C[C@H](O)[C@H]2C]
or a pharmaceutically acceptable salt thereof.

15. A pharmaceutical composition comprising a plurality of nanoparticles comprising a casein, albumin, fibroin, gelatin, or a combination thereof and an active pharmaceutical ingredient that is a compound of the following structure: 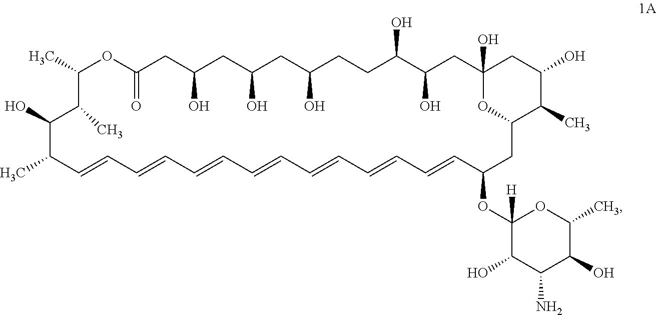 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C]
or a pharmaceutically acceptable salt thereof.

16. The pharmaceutical composition of claim 12 or 15, wherein the pharmaceutically acceptable polymeric excipient is casein.

17. The pharmaceutical composition of claim 12 or 15, wherein the pharmaceutically acceptable polymeric excipient is albumin.

18. The pharmaceutical composition of claim 12 or 15, wherein the pharmaceutically acceptable polymeric excipient is fibroin.

19. The pharmaceutical composition of claim 12 or 15, wherein the pharmaceutically acceptable polymeric excipient is gelatin.

20. The pharmaceutical composition of any one of claims 1 to 19, wherein the pharmaceutical composition is a lyophilized composition.

21. The pharmaceutical composition of any one of claims 1 to 19, further comprising a plurality of microbubbles.

22. A pharmaceutical composition comprising a plurality of microbubbles and a plurality of nanoparticles comprising a compound of the following structure: 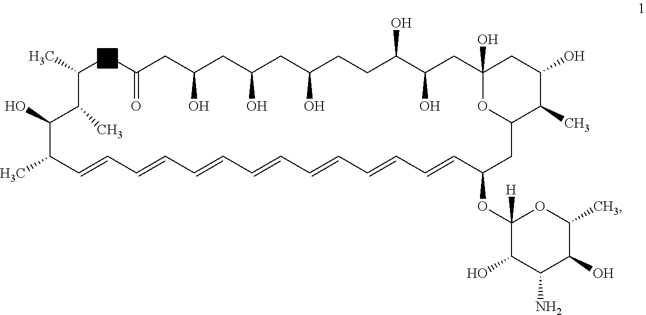 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)CC2O[C@](O)(C[C@@H](O)[C@H](O)CC[C@@H](O)C[C@@H](O)C[C@@H](O)CC(=O)C[C@H]1C)C[C@H](O)[C@H]2C]
or a pharmaceutically acceptable salt thereof.

23. The pharmaceutical composition of claim 22, wherein the compound is 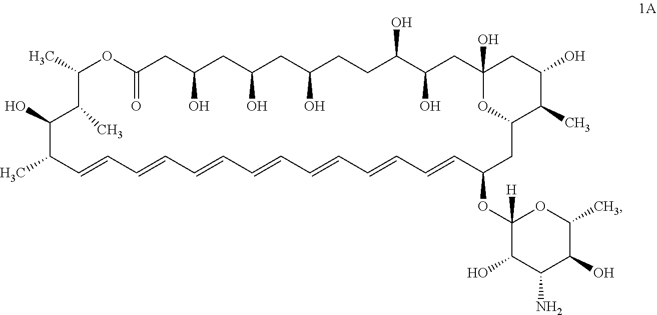 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C]
or a pharmaceutically acceptable salt thereof.

24. The pharmaceutical composition of claim 22 or 23, wherein the nanoparticles comprise a poly(alkyl cyanoacrylate), a polyphosphazene, or a poly(lactic-co-glycolic acid).

25. The pharmaceutical composition of claim 22 or 23, wherein the nanoparticles comprise casein, albumin, fibroin, gelatin, or a combination thereof.

26. The pharmaceutical composition of any one of claims 21 to 25, wherein the microbubbles comprise a perfluorocarbon, hydrocarbon, sulfur fluoride gas, air, a component of air, or a mixture thereof.

27. The pharmaceutical composition of claim 26, wherein the microbubbles comprise nitrogen (N₂), oxygen (O₂), argon (Ar), carbon dioxide (CO₂), helium (He), neon (Ne), methane (CH₄), or a mixture thereof.

28. The pharmaceutical composition of claim 26, wherein the microbubbles comprise a perfluorocarbon.

29. The pharmaceutical composition of claim 26, wherein the microbubbles comprise air or a component thereof.

30. The pharmaceutical composition of any one of claims 21 to 29, wherein the at least a portion of the plurality of nanoparticles is associated with the microbubble surface.

31. The pharmaceutical composition of any one of claims 21 to 30, wherein the pharmaceutical composition further comprises a surface-active protein.

32. The pharmaceutical composition of claim 31, wherein the surface-active protein is an albumin.

33. The pharmaceutical composition of any one of claims 1 to 32, wherein the pharmaceutical composition further comprises a pharmaceutically acceptable surfactant.

34. The pharmaceutical composition of claim 33, wherein the pharmaceutically acceptable surfactant is a non-ionic surfactant.

35. The pharmaceutical composition of claim 33, wherein the pharmaceutically acceptable surfactant is a polyoxyethylene ether, polyoxyethylene fatty acid ester, sorbitan ester, polysorbate, polyethoxylated castor oil, polyoxyethylene/polyoxypropylene block copolymer, or a combination thereof.

36. The pharmaceutical composition of claim 35, wherein the pharmaceutically acceptable surfactant is a polyoxyethylene ether, polyoxyethylene fatty acid ester, or a combination thereof.

37. The pharmaceutical composition of claim 35 or 36, wherein the polyoxyethylene fatty acid ester is a polyoxyethylated 12-hydroxystearic acid.

38. The pharmaceutical composition of claim 35 or 36, wherein the polyoxyethylene ether is a polyoxyethylene lauryl ether.

39. The pharmaceutical composition of any one of claims 1 to 38, wherein the pharmaceutical composition further comprises a pharmaceutically acceptable stabilizer.

40. The pharmaceutical composition of claim 39, wherein the pharmaceutically acceptable stabilizer is vanillin, butylated hydroxytoluene, butylated hydroxyanisole, or vitamin E.

41. The pharmaceutical composition of claim 40, wherein the pharmaceutically acceptable stabilizer is vanillin.

42. The pharmaceutical composition of any one of claims 39 to 41, wherein the pharmaceutical composition comprises 0.1-10% (w/w) of a pharmaceutically acceptable stabilizer relative to the particle mass.

43. The pharmaceutical composition of any one of claims 31 to 33, wherein the pharmaceutical composition comprises 0.5-8% (w/w) of a pharmaceutically acceptable stabilizer relative to the particle mass.

44. The pharmaceutical composition of any one of claims 31 to 33, wherein the pharmaceutical composition comprises 1-5% (w/w) of the pharmaceutically acceptable stabilizer relative to the particle mass.

45. The pharmaceutical composition of any one of claims 1 to 44, wherein the pharmaceutical composition further comprises a pharmaceutically acceptable oil.

46. The pharmaceutical composition of claim 45, wherein the pharmaceutically acceptable oil is selected from the group consisting of medium chain triglycerides, long chain triglycerides, and combinations thereof.

47. The pharmaceutical composition of claim 46, wherein the pharmaceutically acceptable oil is one or more medium chain triglycerides.

48. The pharmaceutical composition of claim 47, wherein the one or more medium chain triglycerides are selected from the group consisting of Miglyol, Captex, and Kollisolv.

49. The pharmaceutical composition of any one of claims 45 to 48, wherein the pharmaceutical composition comprises 0.5-5% (w/w) of a pharmaceutically acceptable oil relative to the particle mass.

50. The pharmaceutical composition of any one of claims 1 to 49, wherein the plurality of nanoparticles has a mean number average diameter of 20-200 nm, as measured by dynamic light scattering.

51. The pharmaceutical composition of any one of claims 1 to 49, wherein the plurality of nanoparticles has a mean number average diameter of 40-100 nm, as measured by dynamic light scattering.

52. The pharmaceutical composition of any one of claims 1 to 49, wherein the plurality of nanoparticles has a mean number average diameter of 30-150 nm, as measured by nanoparticle tracking analysis.

53. The pharmaceutical composition of any one of claims 1 to 49, wherein the plurality of nanoparticles has a mean number average diameter of 80-100 nm, as measured by nanoparticle tracking analysis.

54. The pharmaceutical composition of any one of claims 1 to 53, wherein the pharmaceutical composition is an aqueous composition.

55. The pharmaceutical composition of claim 54, wherein the pH of the pharmaceutical composition is 4.0 to 8.0.

56. The pharmaceutical composition of claim 55, wherein the pH is 5.0 to 7.0.

57. The pharmaceutical composition of any one of claims 1 to 56, wherein the pharmaceutical composition further comprises a co-solvent that is a polar organic solvent.

58. The pharmaceutical composition of claim 57, wherein the polar organic solvent is dimethylsulfoxide, N-methyl-2-pyrrolidone, N,N-dimethylformamide, or a combination thereof.

59. The pharmaceutical composition of any one of claims 1 to 56, wherein the pharmaceutical composition comprises 1-15% dry (w/w) of the active pharmaceutical ingredient, as measured by liquid chromatography.

60. The pharmaceutical composition of any one of claims 1 to 56, wherein the pharmaceutical composition comprises 2-15% dry (w/w) of the active pharmaceutical ingredient, as measured by liquid chromatography.

61. The pharmaceutical composition of any one of claims 1 to 56, wherein the pharmaceutical composition comprises 3-10% dry (w/w) of the active pharmaceutical ingredient, as measured by liquid chromatography.

62. The pharmaceutical composition of any one of claims 1 to 56, wherein the pharmaceutical composition comprises 3.5-10% dry (w/w) of the active pharmaceutical ingredient, as measured by liquid chromatography.

63. The pharmaceutical composition of any one of claims 1 to 56, wherein the pharmaceutical composition comprises 5-10% dry (w/w) of the active pharmaceutical ingredient, as measured by liquid chromatography.

64. The pharmaceutical composition of any one of claims 1 to 63, wherein the pharmaceutical composition comprises 3-6% dry (w/w) of the active pharmaceutical ingredient, as measured by liquid chromatography.

65. A method of treating a subject in need thereof, the method comprising administering to the subject a therapeutically effective amount of the pharmaceutical composition of any one of claims 1 to 64.

66. The method of claim 65, wherein the subject is suffering from a fungal infection caused by Candida, Cryptococcus, Aspergillus, Colletotrichum, Geotrichum, Hormonema, Lecythophora, Paecilomyces, Penicillium, Rhodotorula, Fusarium, Saccharomyces, Trichoderma, Trichophyton, Scopularilopsis, Histoplasma, Blastomyces, or Cocciodioides species.

67. The method of claim 66, wherein the subject is suffering from a fungal infection caused by Candida, Aspergillus, or Cryptococcus spp.

68. The method of claim 67, wherein the subject is suffering from a fungal infection caused by an azole-resistant Aspergillus sp.

69. The method of any one of claims 65 to 67, wherein the pharmaceutical composition is administered intravenously, by inhalation, intranasally, orally, sublingually, buccally, transdermally, intradermally, intramuscularly, intravaginally, parenterally, intra-arterially, intracranially, intrathecally, subcutaneously, intraorbitally, intraventricularly, intraspinally, intraperitoneally, or topically.

70. A method of delivering a therapeutically effective amount of compound 1, compound 1A, or a pharmaceutically acceptable salt thereof, to a target site in a subject, the method comprising administering the pharmaceutical composition of any one of claims 1 to 64 to the subject.

71. The method of claim 70, wherein the pharmaceutical composition is administered intravenously.

72. The method of claim 70 or 71, wherein the target site is the subject's lung.

73. A method of producing a plurality of nanoparticles comprising a pharmaceutically acceptable polymeric excipient and a compound of the following structure: 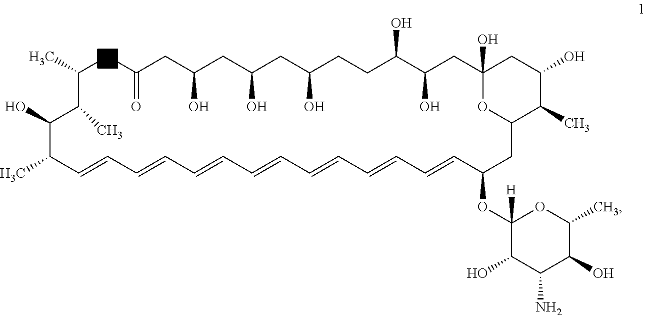 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)CC2O[C@](O)(C[C@@H](O)[C@H](O)CC[C@@H](O)C[C@@H](O)C[C@@H](O)CC(=O)C[C@H]1C)C[C@H](O)[C@H]2C]
or a pharmaceutically acceptable salt thereof;
the method comprising polymerizing a monomeric precursor of the pharmaceutically acceptable polymeric excipient in a liquid comprising the monomeric precursor and the compound or a pharmaceutically acceptable salt thereof, wherein the polymerizing step produces the plurality of nanoparticles.

74. The method of claim 73, wherein the compound is of the following structure: 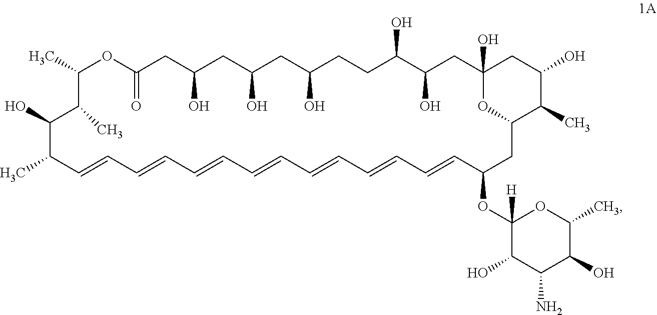 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C]
or a pharmaceutically acceptable salt thereof.

75. The method of claim 73 or 74, wherein the liquid further comprises a pharmaceutically acceptable surfactant.

76. The method of claim 75, wherein the pharmaceutically acceptable surfactant is a non-ionic surfactant.

77. The method of any one of claims 73 to 76, wherein the liquid further comprises a pharmaceutically acceptable stabilizer.

78. The method of any one of claims 73 to 77, wherein the liquid further comprises a pharmaceutically acceptable oil.

79. The method of any one of claims 73 to 78, wherein the monomeric precursor is alkyl cyanoacrylate, and the pharmaceutically acceptable polymeric excipient is poly(alkyl cyanoacrylate).

80. The method of any one of claims 73 to 79, wherein the plurality of nanoparticles has a mean number average diameter of 20-200 nm, as measured by dynamic light scattering.

81. The method of any one of claims 73 to 79, wherein the plurality of nanoparticles has a mean number average diameter of 40-100 nm, as measured by dynamic light scattering.

82. The method of any one of claims 73 to 79, wherein the plurality of nanoparticles has a mean number average diameter of 30-150 nm, as nanoparticle tracking analysis.

83. The method of any one of claims 73 to 79, wherein the plurality of nanoparticles has a mean number average diameter of 80-100 nm, as measured by nanoparticle tracking analysis.

84. The method of any one of claims 73 to 83, wherein the liquid is an aqueous composition.

85. The method of claim 84, wherein the pH of the liquid is 0.5 to 8.0.

86. The method of claim 84, wherein the pH of the liquid is 0.5 to 3.0.

87. The method of claim 84, wherein the pH of the liquid is 2.0 to 8.0.

88. The method of claim 84, wherein the pH of the liquid is 3.0 to 7.0.

89. The method of any one of claims 73 to 88, wherein the method further comprising adding a plurality of microbubbles.

90. The method of any one of claims 73 to 89, wherein the method further comprises lyophilizing the plurality of nanoparticles.

91. The method of any one of claims 73 to 90, wherein the method further comprises dialyzing the plurality of nanoparticles against deionized water.

92. The method of any one of claims 73 to 91, wherein the method further comprises adjusting the pH of the liquid to be in the range 4.0 to 8.0.

93. The method of any one of claims 73 to 92, wherein the method further comprises adjusting the pH of the liquid to be in the range 5.0 to 7.0.

94. The method of claim 92 or 93, wherein the step of adjusting the pH is performed during the step of polymerizing.
